(19) United States
(12) Patent Application Publication
(10) Pub. No.: US 2026/0116843 A1
(43) Pub. Date: Apr. 30, 2026

(54) CRYSTALLINE 4-((L-VALYL)OXY)BUTANOIC ACID SALTS

(71) Applicant: XWPharma Ltd., Grand Cayman (KY)

(72) Inventors: James TIEN, TAOYUAN (TW); Xuan ZHANG, WUHAN (CN); Hao-Wei SHIH, NEW TAIPEI CITY (TW); Jia-Ning XIANG, FREMONT, CA (US)

(21) Appl. No.: 19/364,464

(22) Filed: Oct. 21, 2025

(30) Foreign Application Priority Data
Oct. 29, 2024 (WO) PCT/CN2024/127971

(51) Int. Cl. C07C 229/08

(52) U.S. Cl. CPC C07C 229/08; C07B 2200/13

(57) ABSTRACT

Crystalline 4-((L-valyl)oxy)butanoic acid salts, methods of preparing the crystalline 4-((L-valyl)oxy)butanoic acid salts, pharmaceutical compositions containing the crystalline 4-((L-valyl)oxy)butanoic acid salts, and methods of treatment using the crystalline 4-((L-valyl)oxy)butanoic acid salts are disclosed.

This application claims the benefit under 35 U.S.C. § 119(e) of PCT International Application No. PCT/CN2024/127971 filed on Oct. 29, 2024, which is incorporated by reference in its entirety.

### FIELD

The disclosure relates to crystalline 4-((L-valyl)oxy)butanoic acid salts, methods of preparing the crystalline 4-((L-valyl)oxy)butanoic acid salts, pharmaceutical compositions containing the crystalline 4-((L-valyl)oxy)butanoic acid salts, and methods of treatment using the crystalline 4-((L-valyl)oxy)butanoic acid salts.

### BACKGROUND

4-((L-Valyl)oxy)butanoic acid is a prodrug of γ-hydroxybutyric acid (GHB). GHB is useful for treating sleep disorders such as excessive daytime sleepiness associated with narcolepsy, cataplexy associated with narcolepsy, idiopathic hypersomnia, disturbed nocturnal sleep, and sleep disorders associated with Parkinson's disease.

### SUMMARY

According to the present invention a compound is selected from crystalline 4-((L-valyl)oxy)butanoic acid hydrochloride and crystalline 4-((L-valyl)oxy)butanoic acid oxalate.

According to the present invention a compound is crystalline 4-((L-valyl)oxy)butanoic acid hydrochloride having the structure of Formula (1):

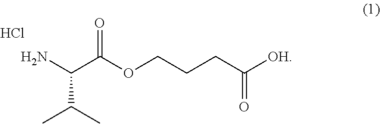

According to the present invention a compound is crystalline 4-((L-valyl)oxy)butanoic acid oxalate having the structure of Formula (2):

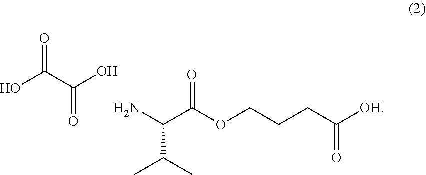

According to the present invention granules comprise a compound according to the present invention.

According to the present invention pharmaceutical compositions comprise a compound according to the present invention or granules according to the present invention.

According to the present invention oral dosage forms comprise a compound according to the present invention, granules according to the present invention, or a pharmaceutical composition according to the present invention.

According to the present invention kits comprise a compound according to the present invention, granules according to the present invention, or a pharmaceutical composition according to the present invention.

According to the present invention methods of treating a disease in a patient comprise administering to a patient in need of such treatment a therapeutically effective amount of a compound according to the present invention, granules according to the present invention, a pharmaceutical composition according to the present invention, or an oral dosage form according to the present invention, wherein the disease is capable of being treated with γ-hydroxybutyric acid.

According to the present invention methods of treating a disease in a patient comprise administering to a patient in need of such treatment a therapeutically effective amount of a compound according to the present invention, granules according to the present invention, a pharmaceutical composition according to the present invention, or an oral dosage form according to the present invention, wherein the disease is selected from narcolepsy, cataplexy, cataplexy with narcolepsy, excessive daytime sleepiness, a sleep disorder associated with Parkinson's disease, idiopathic hypersomnia, disturbed nocturnal sleep, Parkinson's disease, a neurodegenerative disease, sleep disturbance syndrome, fatigue, improving nocturnal sleep, hypnagogic hallucinations, sleep paralysis, fragmented sleep, alcohol withdrawal and dependence, obstructive sleep apnea syndrome, insomnia, insomnia associated with schizophrenia, sleep ignition and maintenance disorders, chronic fatigue syndrome, essential tremor, hemiplegia in patients with alternating hemiplegia of childhood, sedative abuse, and binge eating disorder.

According to the present invention methods of treating fatigue or excessive daytime sleepiness associated with narcolepsy comprise orally administering to a patient in need of such treatment a therapeutically effective amount of a compound according to the present invention, granules according to the present invention, a pharmaceutical composition according to the present invention, or an oral dosage form according to the present invention.

According to the present invention methods of treating narcolepsy, excessive daytime sleepiness, cataplexy, excessive daytime sleepiness associated with narcolepsy, excessive daytime sleepiness associated with Parkinson's disease, excessive daytime sleepiness associated with multiple sclerosis, cataplexy associated with narcolepsy, fatigue, fatigue associated with Parkinson's disease, fatigue associated with multiple sclerosis, or fibromyalgia comprise orally administering to a patient in need of such treatment a therapeutically effective amount of a compound according to the present invention, granules according to the present invention, a pharmaceutical composition according to the present invention or an oral dosage form according to the present invention.

According to the present invention methods of treating a symptom associated with narcolepsy, excessive daytime sleepiness, cataplexy, excessive daytime sleepiness associated with narcolepsy, excessive daytime sleepiness associated with Parkinson's disease, excessive daytime sleepiness associated with multiple sclerosis, cataplexy associated with narcolepsy, fatigue, fatigue associated with Parkinson's diseases, fatigue associated with multiple sclerosis, or fibromyalgia comprise orally administering to a patient in need of such treatment a therapeutically effective amount of a compound according to the present invention, granules according to the present invention, a pharmaceutical composition according to the present invention, or an oral dosage form according to the present invention.

According to the present invention methods of treating an REM sleep behavior disorder, spasmodic dystonia, schizophrenia, insomnia, insomnia associated with schizophrenia, idiopathic hypersomnia, chronic fatigue syndrome, cluster headache, Alzheimer's disease, essential tremor, post-traumatic stress syndrome, insomnia associated with post-traumatic stress syndrome, or anxiety comprise orally administering to a patient in need of such treatment a therapeutically effective amount of a compound according to the present invention, granules according to the present invention, a pharmaceutical composition according to the present invention, or an oral dosage form according to the present invention.

According to the present invention methods of treating a symptom associated with REM sleep behavior disorder, spasmodic dystonia, schizophrenia, insomnia, insomnia associated with schizophrenia, idiopathic hypersomnia, chronic fatigue syndrome, cluster headache, Alzheimer's disease, essential tremor, post-traumatic stress syndrome, insomnia associated with post-traumatic stress syndrome, or anxiety comprise orally administering to a patient in need of such treatment a therapeutically effective amount of a compound according to the present invention, granules according to the present invention, a pharmaceutical composition according to the present invention, or an oral dosage form according to the present invention.

### BRIEF DESCRIPTION OF THE DRAWINGS

The drawings described herein are for illustration purposes only. The drawings are not intended to limit the scope of the present disclosure.

FIG. 1 shows an X-ray powder diffraction (XRPD) pattern of crystalline 4-((L-valyl)oxy)butanoic acid hydrochloride.

FIG. 2 shows a differential scanning calorimetry (DSC) profile of crystalline 4-((L-valyl)oxy)butanoic acid hydrochloride.

FIG. 3 shows an X-ray powder diffraction (XRPD) pattern of crystalline 4-((L-valyl)oxy)butanoic acid oxalate.

### DETAILED DESCRIPTION

For purposes of the following detailed description, it is to be understood that embodiments provided by the present disclosure may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Crystalline 4-((L-valyl)oxy)butanoic acid salts include crystalline 4-((L-valyl)oxy)butanoic acid hydrochloride and crystalline 4-((L-valyl)oxy)butanoic acid oxalate.

“Immediate release” refers to a pharmaceutical composition or a portion of a pharmaceutical composition that releases substantially all of a pharmaceutically active ingredient such as crystalline 4-((L-valyl)oxy)butanoic acid hydrochloride into the gastrointestinal tract of a patient within less than 1 hour following oral administration, such as within less than 50 minutes, within less than 40 minutes, within less than 30 minutes, within less than 20 minutes, or within less than 10 minutes following oral administration. For example, an immediate release dosage form can release greater than 90%, greater than 95%, or greater than 98% of the pharmaceutically active ingredient in the pharmaceutical composition into the gastrointestinal tract within less than 1 hour such as within less than 50 minutes, less than 40 minutes, less than 30 minutes, less than 20 minutes, or less than 10 minutes, following oral administration. Immediate release pharmaceutical compositions can be used to administer pharmaceutically active ingredients that are absorbed into the systemic circulation from the upper portion of the gastrointestinal tract.

“Modified release” pharmaceutical compositions include controlled release formulations, delayed release formulations, extended-release formulations, sustained release formulations, timed release formulations, pulsatile release formulations, and pH-dependent release formulations. These formulations are intended to release a pharmaceutically active ingredient from the pharmaceutical composition at a desired rate and/or at a desired time following oral administration by a patient and/or at a certain location or locations within the gastrointestinal tract and/or at a certain pH within the gastrointestinal tract. The USP defines a modified release system as one in which the time course or location of drug release or both, are chosen to accomplish objectives of therapeutic effectiveness or convenience not fulfilled by immediate release dosage forms. A modified release oral dosage form can include extended release and delayed-release components. A delayed release dosage form is one that releases a drug all at once at a time other than promptly after administration. A modified release formulation can include delayed-release using enteric coatings, site-specific or timed release such as for colonic delivery, extended-release formulations including, for example, formulations capable of providing zero-order, first-order, or biphasic release profiles, and programmed release such as pulsatile and delayed extended release.

“Sustained release” pharmaceutical compositions and coatings provide for a dissolution rate over an extended period of time following oral administration. Granules having a sustained release coating can be referred to as sustained release granules. A pharmaceutical composition comprising a sustained release granules can be referred to as a sustained release pharmaceutical composition.

“pH-release” pharmaceutical compositions and coatings provide for an increased dissolution rate at an intended pH.

“Pulsatile release” pharmaceutical compositions and coatings exhibit an increased dissolution rate at intervals, where the release intervals can be determined by time, exposure to internal stimuli, or exposure to external stimuli. Examples of pulsatile-release systems include capsular systems, osmotic systems, systems having erodible membranes, and systems having a rupturable coating. Examples of stimuli include temperature, chemicals, electrical stimuli, and magnetic stimuli.

“Timed-release” pharmaceutical compositions and coatings have a dissolution rate that is a function of time. A time-release pharmaceutical composition or coating includes, for example, delayed release, sustained release, and/or extended-release pharmaceutical compositions and coatings.

“Delayed release” pharmaceutical compositions and coatings provide for an increased dissolution rate at an intended time after administration.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical granules” refer to granules comprising a 4-((L-valyl)oxy)butanoic acid salt provided by the present disclosure that are suitable for oral administration and/or that are provided to a patient for combination with a liquid for oral administration. Pharmaceutical granules are not configured to be compressed into a tablet dosage form.

“Pharmaceutical composition” refers to a composition comprising a crystalline 4-((L-valyl)oxy)butanoic acid salt and at least one pharmaceutically acceptable vehicle, with which the crystalline 4-((L-valyl)oxy)butanoic acid salt is administered to a patient. Pharmaceutically acceptable vehicles are known in the art.

“Disease” refers to a disease, disorder, condition, or symptom of any of the foregoing.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). In some embodiments, “preventing” or “prevention” refers to reducing symptoms of the disease by administering a compound provided by the present disclosure in a preventative fashion. The application of a therapeutic agent for preventing or prevention of a disease of disorder is known as ‘prophylaxis.’ Compounds provided by the present disclosure can provide superior prophylaxis because of lower long-term side effects over long time periods.

“Prodrug” refers to a derivative of a drug molecule that requires a transformation within the body to release the active drug. Prodrugs are frequently, although not necessarily, pharmacologically inactive until converted to the parent drug. Prodrugs may be obtained by bonding a promoiety typically via a functional group, to a drug. For example, referring to 4-((L-valyl)oxy)butanoic acid, a NH₂—CH(—CH₃)₂)—C(O)— promoiety is bonded to γ-hydroxybutyric acid. 4-((L-Valyl)oxy)butanoic acid is a prodrug of γ-hydroxybutyric acid that can be metabolized within a patient's body to release γ-hydroxybutyric acid. A crystalline 4-((L-valyl)oxy)butanoic acid salt is a prodrug of γ-hydroxybutyric acid that can be metabolized within a patient's body to release γ-hydroxybutyric acid.

“Promoiety” refers to a group bonded to a drug, typically to a functional group of the drug, via a bond or bonds that are cleavable under specified conditions of use. The bond or bonds between the drug and promoiety may be cleaved by enzymatic or non-enzymatic processes. Under the conditions of use, for example following administration to a patient, the bond or bonds between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature or pH. The agent may be endogenous to the conditions of use, such as an enzyme present in the systemic circulation of a patient to which the prodrug is administered or the acidic conditions of the stomach or the agent may be supplied exogenously. Compounds provided by the present disclosure are prodrugs of γ-hydroxybutyric acid. The promoiety of 4-((L-valyl)oxy)butanoic acid has the structure:

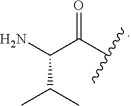 [SMILES: *C(=O)[C@@H](N)C(C)C]

The promoiety is cleaved in vivo to release γ-hydroxybutyric acid in the systemic circulation of a patient.

“Curing” a disease refers to eliminating a disease or disorder or eliminating a symptom of a disease or disorder.

“Sealed storage stability” refers to the stability of a compound that is enclosed within two sealed polyethylene bags with a desiccant material placed between the two polyethylene bags and the two sealed polyethylene bags are enclosed within a sealed one-layer aluminum bag.

“Treating” or “treatment” of a disease or disorder refers to inhibiting the disease or disorder or one or more clinical symptoms of the disease or disorder, arresting the development of the disease or disorder or one or more clinical symptoms of the disease or disorder, relieving the disease or disorder or one or more clinical symptoms of the disease or disorder, causing the regression of the disease or disorder or one or more clinical symptoms of the disease or disorder, reducing the severity of one or more clinical symptom of the disease or disorder, delaying the onset of one or more clinical symptoms of the disease or disorder, mitigating one or more clinical symptoms of the disease or disorder and/or stabilizing the disease or disorder or one or more clinical symptoms of the disease or disorder. “Treating” or “treatment” of a disease or disorder includes producing a clinically beneficial effect without curing the underlying disease or disorder.

“Therapeutically effective amount” refers to the amount of a compound such as pharmaceutically active ingredient that, when administered to a patient for treating a disease, or at least one of the clinical symptoms of a disease, is sufficient to affect such treatment of the disease or symptom thereof. A “therapeutically effective amount” may vary depending, for example, on the compound, the disease and/or symptoms of the disease, the severity of the disease and/or symptoms of the disease or disorder, the age, weight, and/or health of the patient to be treated, and the judgment of the prescribing physician. A therapeutically effective amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

Bulk density can be determined according to USP 616, Method 1.

Tapped bulk density can be determined according to USP 616.

Specific surface area can be determined by laser diffraction.

The Hausner Ratio can be determined according to USP 1174.

The parameter D90 refers to the point in the size distribution of a sample, up to and including which 90% of the total volume of material in the sample is contained. For example, for a D90 of 400 μm, 90% of the sample volume has a size of 400 μm or less D50 is the size below which 50% of the total volume of material in the sample is contained. Similarly, D10 refers to the size below which 10% of the total volume of material in the sample is contained. The volume distribution of the sample can be determined by laser diffraction or by sieve analysis.

Equivalents of γ-hydroxybutyric acid such as gram-equivalents of γ-hydroxybutyric acid refers to the grams of γ-hydroxybutyric acid in an amount of crystalline 4-((L-valyl)oxy)butanoic acid hydrochloride. The amount of gram-equivalents of γ-hydroxybutyric acid can be determined by multiplying the amount in grams of crystalline 4-((L-valyl)oxy)butanoic acid hydrochloride by 0.435. For example, 10 grams of 4-((L-valyl)oxy)butanoic acid hydrochloride corresponds to 4.35 gram-equivalents of γ-hydroxybutyric acid.

Reference is now made to crystalline 4-((L-valyl)oxy)butanoic acid salts, methods of making crystalline 4-((L-valyl)oxy)butanoic acid salts, pharmaceutical compositions comprising crystalline 4-((L-valyl)oxy)butanoic acid salts, and uses of crystalline 4-((L-valyl)oxy)butanoic acid salts. The disclosed crystalline 4-((L-valyl)oxy)butanoic acid salts, pharmaceutical compositions, methods, and uses are not intended to be limiting of the claims. To the contrary, the claims are intended to cover all alternatives, modifications, and equivalents.

4-((L-Valyl)oxy)butanoic acid salts include crystalline 4-((L-valyl)oxy)butanoic acid hydrochloride and crystalline 4-((L-valyl)oxy)butanoic acid oxalate.

A crystalline 4-((L-valyl)oxy)butanoic acid salt can consist of crystalline 4-((L-valyl)oxy)butanoic acid hydrochloride and crystalline 4-((L-valyl)oxy)butanoic acid oxalate.

A crystalline 4-((L-valyl)oxy)butanoic acid salt can be crystalline 4-((L-valyl)oxy)butanoic acid hydrochloride.

A crystalline 4-((L-valyl)oxy)butanoic acid salt can be crystalline 4-((L-valyl)oxy)butanoic acid oxalate.

Crystalline 4-((L-valyl)oxy)butanoic acid hydrochloride, is a stable crystalline form of 4-((L-valyl)oxy)butanoic acid hydrochloride. Crystalline 4-((L-valyl)oxy)butanoic acid hydrochloride has the structure of Formula (1):

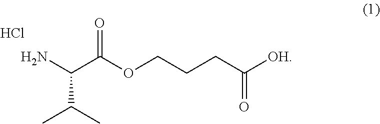

4-((L-Valyl)oxy)butanoic acid hydrochloride is also referred to as (S)-4-(2-amino-3-methylbutanoyloxy)butanoic acid hydrochloride.

4-((L-Valyl)oxy)butanoic acid hydrochloride can be prepared as described in Example 1.

Crystalline 4-((L-Valylvalyl)oxy)butanoic acid hydrochloride can be prepared as described in Example 1.

Crystalline 4-((L-valyl)oxy)butanoic acid hydrochloride can be characterized by an X-ray powder diffraction (XRPD) pattern comprising characteristic diffraction peaks at least at 8.05°±0.20°, 21.66°±0.20°, and 24.09°±0.20° expressed as 2θ/θ angles determined using Cu-Kα radiation.

Crystalline 4-((L-valyl)oxy)butanoic acid hydrochloride can be characterized by an XRPD pattern comprising characteristic diffraction peaks at least at 8.05°±0.10°, 21.66°±0.10°, and 24.09°±0.100 expressed as 2θ/θ angles determined using Cu-Kα radiation.

Crystalline 4-((L-valyl)oxy)butanoic acid hydrochloride can be characterized by an XRPD pattern comprising characteristic diffraction peaks at least at 8.05°±0.20°, 10.72°±0.20°, 16.12°±0.20°, 21.66°±0.20°, 24.09°±0.20°, and 27.38°±0.20° expressed as 2θ/θ angles determined using Cu-Kα radiation.

Crystalline 4-((L-valyl)oxy)butanoic acid hydrochloride can be characterized by an XRPD pattern comprising characteristic diffraction peaks at least at 8.05°±0.10°, 10.72°±0.10°, 16.12°±0.10°, 21.66°±0.10°, 24.09°±0.10°, and 27.38°±0.10° expressed as 2θ/θ angles determined using Cu-Kα radiation.

Crystalline 4-((L-valyl)oxy)butanoic acid hydrochloride can be characterized by an XRPD pattern comprising characteristic diffraction peaks at least at 8.05°±0.20°, 10.72±0.20°, 16.12°±0.20°, 16.53±0.20°, 17.62°±0.20°, 21.66°±0.20°, 24.09°±0.20°, 26.03°±0.20°, 27.38°±0.20°, and 30.09°±0.20° expressed as 2θ/θ angles determined using Cu-Kα radiation.

Crystalline 4-((L-valyl)oxy)butanoic acid hydrochloride can be characterized by an XRPD pattern comprising characteristic diffraction peaks at least at 8.05°±0.10°, 10.72±0.10°, 16.12°±0.10°, 16.53±0.10°, 17.62°±0.10°, 21.66°±0.10°, 24.09°±0.10°, 26.03°±0.10°, 27.38°±0.10°, and 30.09°±0.100 expressed as 2θ/θ angles determined using Cu-Kα radiation.

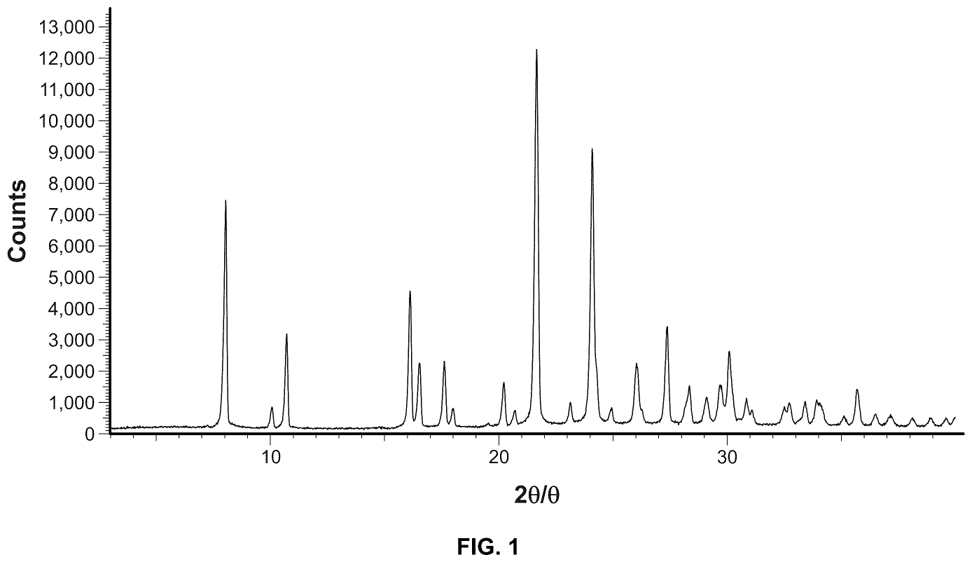

Crystalline 4-((L-valyl)oxy)butanoic acid hydrochloride can be characterized by an XRPD pattern as substantially shown in FIG. 1.

Crystalline 4-((L-valyl)oxy)butanoic acid hydrochloride can have a differential scanning calorimetry (DSC) profile characterized by an exotherm with an onset temperature at 97.04° C.±2° C., and a peak at 99.19° C.±2° C.

Crystalline 4-((L-valyl)oxy)butanoic acid hydrochloride can have a DSC profile characterized by an exotherm with an onset temperature at 97.04° C.±1° C., and a peak temperature at 99.19° C.±1° C., or an onset temperature at 97.04° C.±5° C., and a peak at 99.19° C.±5° C.

Crystalline 4-((L-valyl)oxy)butanoic acid hydrochloride can have a DSC profile characterized by an exotherm with an onset temperature at 97.04° C., and a peak temperature at 99.19° C.

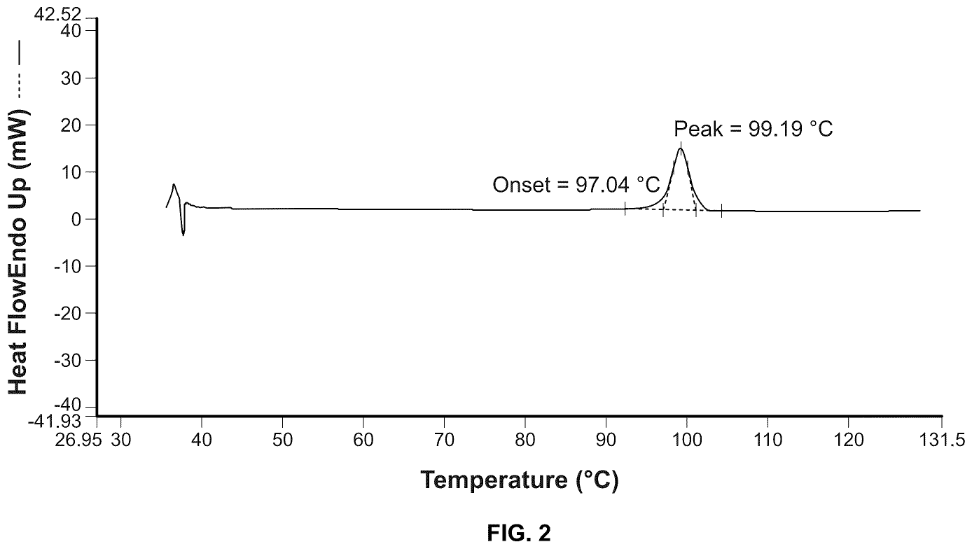

Crystalline 4-((L-valyl)oxy)butanoic acid hydrochloride can have a DSC profile substantially as shown in FIG. 2.

Crystalline 4-((L-valyl)oxy)butanoic acid hydrochloride is stable in an aqueous solution at a pH from 1.2 to 8.0 at a temperature of 37° C., for up to 240 hours and/or in dimethyl sulfoxide (DMSO) for up to 96 hours.

Crystalline 4-((L-valyl)oxy)butanoic acid hydrochloride is substantially stable under sealed storage conditions.

For example, following one month, three months, and/or six months of sealed storage at 25° C./60% RH crystalline 4-((L-valyl)oxy)butanoic acid hydrochloride can have a water content, for example of less than 5 wt %, less than 4 wt %, less than 3 wt %, less than 2 wt %, less than 1 wt %, less than 0.8 wt %, less than 0.6 wt %, less than 0.4 wt %, less than 0.2 wt %, or less than 0.1 wt %, where wt % is based on the total weight of 4-((L-valyl)oxy)butanoic acid and water in the crystalline 4-((L-valyl)oxy)butanoic acid hydrochloride, and the water content is determined using Karl Fischer analysis.

For example, after 36 months of sealed storage at 25° C./65% RH the water content can be less than 2.5 wt % or less than 2.0 wt %, where wt % is based on the total weight of the sample of crystalline 4-((L-valyl)oxy)butanoic acid hydrochloride.

Crystalline 4-((L-valyl)oxy)butanoic acid hydrochloride can have a compositional purity, for example, of greater than 95 wt %, greater than 96 wt %, greater than 97 wt %, greater than 98 wt %, greater than 99 wt %, greater than 99.2 wt %, greater than 99.4 wt %, greater than 99.6 wt %, greater than 99.8 wt %, or greater than 99.9 wt %, where wt % is based on the total weight of the sample of crystalline 4-((L-valyl)oxy)butanoic acid hydrochloride, where the compositional purity is determined using high-pressure liquid chromatography (HPLC).

Crystalline 4-((L-valyl)oxy)butanoic acid hydrochloride can have an enantiomeric impurity content, for example, of less than 5 wt %, less than 4 wt %, less than 3 wt %, less than 2 wt %, less than 1 wt %, less than 0.8 wt %, less than 0.6 wt %, less than 0.4 wt %, less than 0.2 wt %, or less than 0.1 wt %, where wt % is based on the total weight of the sample of crystalline 4-((L-valyl)oxy)butanoic acid hydrochloride, where the enantiomeric impurity content is determined using liquid chromatography-mass spectroscopy (LC-MS).

Crystalline 4-((L-valyl)oxy)butanoic acid hydrochloride can have an enantiomeric purity, for example, of greater than 98 wt %, greater than 99 wt %, greater than 99.2 wt %, greater than 99.4 wt %, greater than 99.6 wt %, greater than 99.8 wt %, or greater than 99.9 wt %, where wt % is based on the total weight of the sample of crystalline 4-((L-valyl)oxy)butanoic acid hydrochloride, where the enantiomeric purity is determined using HPLC.

Following 1 month, 3 months, and/or 6 months of storage at 25° C./65% RH crystalline 4-((L-valyl)oxy)butanoic acid hydrochloride can have an enantiomeric impurity content, for example, of less than 5 wt %, less than 4 wt %, less than 3 wt %, less than 2 wt %, less than 1 wt %, less than 0.8 wt %, less than 0.6 wt %, less than 0.4 wt %, less than 0.2 wt %, or less than 0.1 wt %, where wt % is based on the total weight of the sample of crystalline 4-((L-valyl)oxy)butanoic acid hydrochloride, and enantiomeric impurity content is determined using HPLC

The impurity content of crystalline 4-((L-valyl)oxy)butanoic acid hydrochloride can be determined using high pressure liquid chromatography/mass spectroscopy.

Methods of synthesizing 4-((L-valyl)oxy)butanoic acid and properties of 4-((L-valyl)oxy)butanoic acid are disclosed, for example, in U.S. Pat. No. 10,457,627 and in U.S. Pat. No. 11,279,669, each of which is incorporated by reference in its entirety. Methods of synthesizing 4-((L-valyl)oxy)butanoic acid are also disclosed in Example 1.

For example, 4-((L-valyl)oxy)butanoic acid can be prepared by: (a) contacting benzyl bromide with butyrolactone to provide benzyl 4-hydroxybutanoate (2a); (b) contacting benzyl 4-hydroxybutanoate (2a) with carbobenzyloxy-L-valine to provide 4-(benzoyloxy)-4-oxobutyl((benzoyloxy)carbonyl)-L-valinate (2b); and (c) deprotecting 4-(benzoyloxy)-4-oxobutyl((benzoyloxy)carbonyl)-L-valinate (2b) to provide 4-((L-valyl)oxy)butanoic acid.

Alternatively, 4-((L-valyl)oxy)butanoic acid can be prepared according to the method shown in Scheme 1:

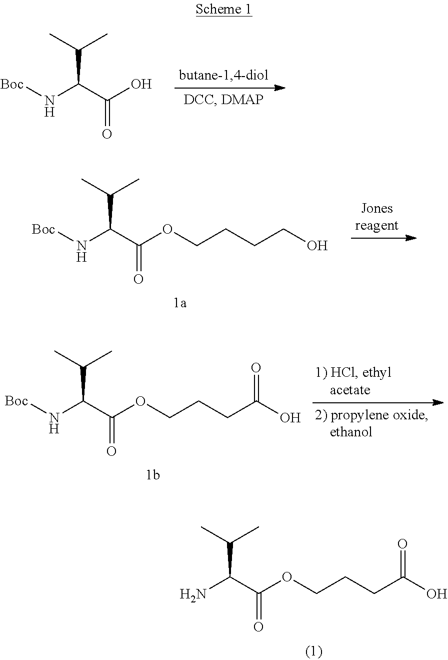 [SMILES: CC(C)[C@H](NC(=O)OC(C)(C)C)C(=O)O ; CC(C)[C@H](NC(=O)OC(C)(C)C)C(=O)OCCCCO ; CC(C)[C@H](NC(=O)OC(C)(C)C)C(=O)OCCCC(=O)O ; CC(C)[C@H](N)C(=O)OCCCC(=O)O]

Boc-L-valine may be coupled to butane-1,4-diol to provide (S)-4-hydroxybutyl 2-(tert-butoxycarbonylamino)-3-methylbutanoate (1a) using a peptide coupling reagent, such as N,N′-dicyclohexylcarbodiimide (DCC), BOP, DEPBT, N,N′-diisopropylcarbodiimide (DIC), 1-ethyl-3-(3-dimethylaminopropyl)carbodiimide (EDCI), 4-(4,6-dimethoxy-1,3,5-triazin-2-yl)-4-methylmorpholinium chloride (DMTMM), HATU, HBTU, TBTU, PyAOP, PyBOP, 1,1′-thiocarbonyldiimdazole (TCDI), or 1,1′-carbonyldiimidazole (CDI). 1-Hydroxybenzotriazole (HOBt) or 1-hydroxy-7-aza-benzotriazole (HOAt) can be included to suppress racemization.

(S)-4-Hydroxybutyl 2-(tert-butoxycarbonylamino)-3-methylbutanoate (1a) can be treated with an oxidizer to provide (S)-4-(2-(tert-butoxycarbonylamino)-3-methylbutanoyloxy)butanoic acid (1b). Examples of suitable oxidizers include Jones reagent, potassium permanganate, ruthenium tetroxide, pyridinium dichromate in dimethylformamide (DMF), tetrapropylammonium perruthenate, or catalytic tetrapropylammonium perruthenate with stoichiometric N-methylmorpholine N-oxide. An alcohol can be oxidized to the corresponding aldehyde, such as with Dess-Martin periodinane, and subsequently oxidized to the carboxylic acid, such as with sodium chlorite.

(S)-4-(2-(tert-Butoxycarbonylamino)-3-methylbutanoyloxy)butanoic acid (1b) can be treated with an acid to provide the salt form of compound (1b). Examples of suitable acids include HCl, HCl in ethyl acetate, aqueous HCl, HCl in 1,4-dioxane, and trifluoroacetic acid. The salt form of compound (1b) can be neutralized with an acid scavenger to provide the free amine form of 4-((L-valyl)oxy)butanoic acid. Examples of suitable acid scavengers include propylene oxide, an organic base, an amine base, an inorganic base, a carbonate salt, or a hydroxide salt.

Crystalline 4-((L-valyl)oxy)butanoic acid hydrochloride can be prepared by (i) dissolving 4-((L-valyl)oxy)butanoic acid hydrochloride in a first solvent to obtain a solution; and (ii) crystallizing the solution to obtain crystalline 4-((L-valyl)oxy)butanoic acid hydrochloride.

The first solvent can comprise, for example, methanol, ethanol, isopropanol, tetrahydrofuran, ethyl acetate, acetone, isobutanol, isopropyl acetate, acetonitrile, 2-butanone, toluene, water, tert-butyl methyl ether, n-propanol, isopentanol, butyl acetate, ethyl formate, methyl acetate, isobutyl acetate, n-heptane, dichloromethane, 1,4-dioxane, cyclohexane, xylene, 4-methyl-2-pentanone, ethyl ether, or a combination of any of the foregoing.

4-((L-Valyl)oxy)butanoic acid hydrochloride can be dissolved in the solvent at a temperature, for example, from 60° C. to 90° C., from 65° C. to 85° C., or from 70° C. to 80° C.

After dissolving 4-((L-valyl)oxy)butanoic acid hydrochloride in a first solvent, a second solvent can be added to the solution. For example, the second solvent can comprise acetonitrile, tetrahydrofuran, isopropanol, acetone, ethyl acetate, tert-butyl methyl ether, 1,4-dioxane, or a combination of any of the foregoing.

Crystallizing 4-((L-valyl)oxy)butanoic acid hydrochloride can comprise, for example, heating the solution to a temperature from 60° C. to 90° C., such as from 65° C. to 85° C., or from 60° C. to 80° C., and maintaining the solution at this temperature, for example, from 0.5 hours to 2 hours.

Crystallizing can further comprise cooling the heated solution, for example, to a temperature from 20° C. to 25° C. to provide crystalline 4-((L-valyl)oxy)butanoic acid hydrochloride

The crystallized 4-((L-valyl)oxy)butanoic acid hydrochloride can be filtered and washed to provide crystalline 4-((L-valyl)oxy)butanoic acid hydrochloride.

Crystalline 4-((L-valyl)oxy)butanoic acid hydrochloride can be recrystallized, for example, by warming a solvent, adding crystalline 4-((L-valyl)oxy)butanoic acid to the solvent, and dissolving the crystalline 4-((L-valyl)oxy)butanoic acid in the solvent. The solvent can be, for example, ethanol, 50:1 ethanol/water, water, methanol, or isopropanol. Additional solvent can be added and/or optionally a suitable amount of an anti-solvent can be added to the clear solution. Examples of suitable anti-solvent include acetone ethyl acetate and methyl tert-butyl ether. For example, the volume ratio of methanol to methyl tert-butyl ether can be bout 1:3. After the solution is stirred for about 30 minutes, the solution can be cooled to a temperature from 20° C. to 25° C. and filtered to provide crystalline 4-((L-valyl)oxy)butanoic acid hydrochloride as a solid precipitate.

Crystalline 4-((L-valyl)oxy)butanoic acid oxalate is a stable crystalline form of 4-((L-valyl)oxy)butanoic acid oxalate. Crystalline 4-((L-valyl)oxy)butanoic acid oxalate has the structure of Formula (2):

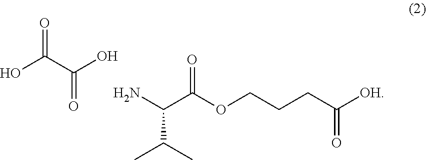

4-((L-Valyl)oxy)butanoic acid oxalate is also referred to as (S)-4-(2-amino-3-methylbutanoyloxy)butanoic acid oxalate.

4-((L-Valyl)oxy)butanoic acid oxalate can be characterized by an XRPD pattern comprising characteristic diffraction peaks at least at 6.48°±0.20°, 9.44°±0.20°, 18.20°±0.20°, and 27.24°±0.20°, expressed as 2θ/θ angles determined using Cu-Kα radiation.

4-((L-Valyl)oxy)butanoic acid oxalate can be characterized by an XRPD pattern comprising characteristic diffraction peaks at least at 6.48°±0.10°, 9.44°±0.10°, 18.20°±0.10°, and 27.24°±0.10°, expressed as 2θ/θ angles determined using Cu-Kα radiation.

4-((L-Valyl)oxy)butanoic acid oxalate can be characterized by an XRPD pattern comprising characteristic diffraction peaks at least at 6.48°±0.20°, 9.44°±0.20°, 11.04°±0.20°, 17.03°±0.20°, 18.20°±0.20°, 27.24°±0.20°, and 28.86°±0.20°, expressed as 2θ/θ angles determined using Cu-Kα radiation.

4-((L-Valyl)oxy)butanoic acid oxalate can be characterized by an XRPD pattern comprising characteristic diffraction peaks at least at 6.48°±0.20°, 9.44°±0.10°, 11.04°±0.10°, 17.03°±0.10°, 18.20°±0.10°, 27.24°±0.10°, and 28.86°±0.10°, expressed as 2θ/θ angles determined using Cu-Kα radiation.

4-((L-Valyl)oxy)butanoic acid oxalate can be characterized by an XRPD pattern comprising characteristic diffraction peaks at least at 6.48°±0.20°, 9.44°±0.20°, 11.04°±0.20°, 17.03°±0.20°, 18.20°±0.20°, 18.73°±0.20°, 19.08°±0.20°, 21.73°±0.20°, 22.46°±0.20°, 27.24°±0.20°, 28.86°±0.20°, and 29.45°±0.20°, expressed as 2θ/θ angles determined using Cu-Kα radiation.

4-((L-Valyl)oxy)butanoic acid oxalate can be characterized by an XRPD pattern comprising characteristic diffraction peaks at least at 6.48°±0.10°, 9.44°±0.10°, 11.04°±0.10°, 17.03°±0.10°, 18.20°±0.10°, 18.73°±0.10°, 19.08°±0.10°, 21.73°±0.10°, 22.46°±0.10°, 27.24°±0.10°, 28.86°±0.10°, and 29.45°±0.10°, expressed as 2θ/θ angles determined using Cu-Kα radiation.

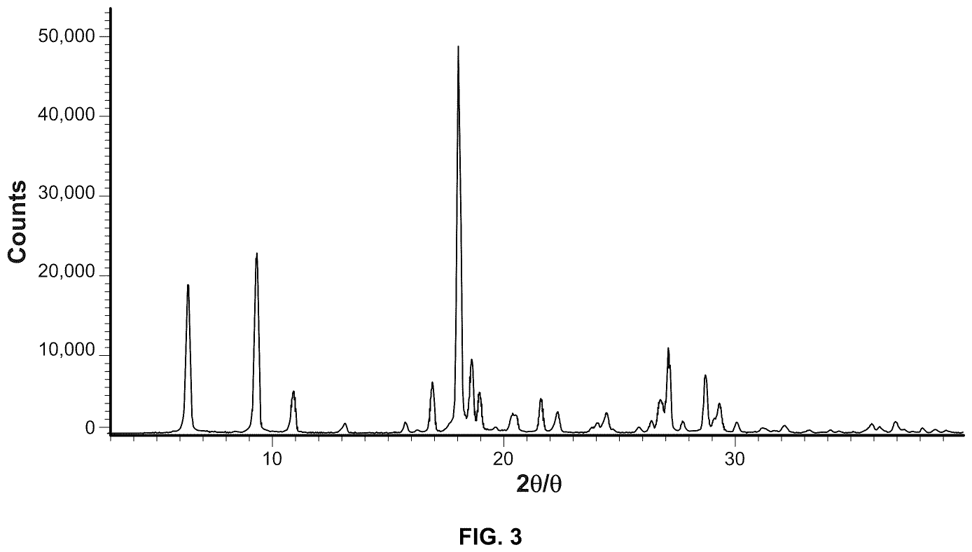

4-((L-Valyl)oxy)butanoic acid oxalate can be characterized by an XRPD profile substantially as shown in FIG. 3.

Crystalline 4-((L-Valyl)oxy)butanoic acid oxalate can be prepared as described in Example 2.

Crystalline 4-((L-valyl)oxy)butanoic acid oxalate can be stable in an aqueous solution at a pH from 1.2 to 8.0 at a temperature of 37° C., for up to 240 hours or in dimethyl sulfoxide (DMSO) for up to 96 hours.

Crystalline 4-((L-valyl)oxy)butanoic acid oxalate can be substantially stable under sealed storage conditions.

For example, following one month, three months and/or six months of sealed storage at 25° C./60% RH crystalline 4-((L-valyl)oxy)butanoic acid oxalate can have a water content, for example of less than 5 wt %, less than 4 wt %, less than 3 wt %, less than 2 wt %, less than 1 wt %, less than 0.8 wt %, less than 0.6 wt %, less than 0.4 wt %, less than 0.2 wt %, or less than 0.1 wt %, where wt % is based on the total weight of 4-((L-valyl)oxy)butanoic acid oxalate and water in the crystalline 4-((L-valyl)oxy)butanoic acid oxalate, and the water content is determined using Karl Fischer analysis.

For example, after 36 months of sealed storage at 25° C./65% RH the water content can be less than 2.5 wt % or less than 2.0 wt %, where wt % is based on the total weight of the sample of crystalline 4-((L-valyl)oxy)butanoic acid oxalate.

Crystalline 4-((L-valyl)oxy)butanoic acid oxalate can have a compositional purity, for example, of greater than 95 wt %, greater than 96 wt %, greater than 97 wt %, greater than 98 wt %, greater than 99 wt %, greater than 99.2 wt %, greater than 99.4 wt %, greater than 99.6 wt %, greater than 99.8 wt %, or greater than 99.9 wt %, where wt % is based on the total weight of the sample of crystalline 4-((L-valyl)oxy)butanoic acid oxalate, and compositional purity is determined using HPLC.

Crystalline 4-((L-valyl)oxy)butanoic acid oxalate can have an impurity content, for example, of less than 5 wt %, less than 4 wt %, less than 3 wt %, less than 2 wt %, less than 1 wt %, less than 0.8 wt %, less than 0.6 wt %, less than 0.4 wt %, less than 0.2 wt %, or less than 0.1 wt %, where wt % is based on the total weight of the sample of crystalline 4-((L-valyl)oxy)butanoic acid oxalate, and the impurity content is determined using HPLC.

Crystalline 4-((L-valyl)oxy)butanoic acid oxalate can have an enantiomeric purity, for example, of greater than 98 wt %, greater than 99 wt %, greater than 99.2 wt %, greater than 99.4 wt %, greater than 99.6 wt %, greater than 99.8 wt %, or greater than 99.9 wt %, where wt % is based on the total weight of the sample of crystalline 4-((L-valyl)oxy)butanoic acid oxalate, and the enantiomeric purity is determined using HPLC.

Following 1 month, 3 months and/or 6 months of storage at 25° C./65% RH crystalline 4-((L-valyl)oxy)butanoic acid oxalate can have an enantiomeric impurity content, for example, of less than 5 wt %, less than 4 wt %, less than 3 wt %, less than 2 wt %, less than 1 wt %, less than 0.8 wt %, less than 0.6 wt %, less than 0.4 wt %, less than 0.2 wt %, or less than 0.1 wt %, where wt % is based on the total weight of the sample of crystalline 4-((L-valyl)oxy)butanoic acid oxalate, and the enantiomeric impurity content is determined using HPLC.

The impurity content of crystalline 4-((L-valyl)oxy)butanoic acid oxalate can be determined using HPLC.

Crystalline 4-((L-valyl)oxy)butanoic acid oxalate can be prepared by reacting 4-((L-valyl)oxy)butanoic acid and oxalic acid in methanol in the presence of methyl tert-butyl ether for up to 20 hours at 25° C., and filtering, washing and drying the white precipitate.

Crystalline 4-((L-valyl)oxy)butanoic acid oxalate can be prepared by (i) dissolving 4-((L-valyl)oxy)butanoic acid oxalate in a first solvent to obtain a solution; and (ii) crystallizing the solution to obtain crystalline 4-((L-valyl)oxy)butanoic acid oxalate.

The first solvent can comprise, for example, methanol, ethanol, isopropanol, tetrahydrofuran, ethyl acetate, acetone, isobutanol, isopropyl acetate, acetonitrile, 2-butanone, toluene, water, tert-butyl methyl ether, n-propanol, isopentanol, butyl acetate, ethyl formate, methyl acetate, isobutyl acetate, n-heptane, dichloromethane, 1,4-dioxane, cyclohexane, xylene, 4-methyl-2-pentanone, ethyl ether, or a combination of any of the foregoing.

4-((L-Valyl)oxy)butanoic acid oxalate can be dissolved in the solvent at a temperature, for example, from 60° C. to 90° C., from 65° C. to 85° C., or from 70° C. to 80° C.

After dissolving 4-((L-valyl)oxy)butanoic acid oxalate in a first solvent, a second solvent can be added to the solution. For example, the second solvent can comprise acetonitrile, tetrahydrofuran, isopropanol, acetone, ethyl acetate, tert-butyl methyl ether, 1,4-dioxane, or a combination of any of the foregoing.

Crystallizing 4-((L-valyl)oxy)butanoic acid oxalate can comprise, for example, heating the solution to a temperature from 60° C. to 90° C., such as from 65° C. to 85° C., or from 60° C. to 80° C., and maintaining the solution at this temperature, for example, from 0.5 hours to 2 hours.

Crystallizing can further comprise cooling the heated solution, for example, to a temperature from 20° C. to 25° C.

The crystallized 4-((L-valyl)oxy)butanoic acid oxalate can be filtered and washed to provide crystalline 4-((L-valyl)oxy)butanoic acid oxalate.

Crystalline 4-((L-valyl)oxy)butanoic acid oxalate can be recrystallized, for example, by warming a solvent, adding crystalline 4-((L-valyl)oxy)butanoic acid oxalate to the solvent, and dissolving the crystalline 4-((L-valyl)oxy)butanoic acid oxalate in the solvent. The solvent can be, for example, ethanol, 50:1 ethanol/water, water, methanol, or isopropanol. Additional solvent can be added and/or optionally a suitable amount of an anti-solvent can be added to the clear solution. Examples of suitable anti-solvent include acetone ethyl acetate and methyl tert-butyl ether. For example, the volume ratio of methanol to methyl tert-butyl ether can be bout 1:3. After the solution is stirred for about 30 minutes, the solution can be cooled to a temperature from 20° C. to 25° C. and filtered to provide crystalline 4-((L-valyl)oxy)butanoic acid oxalate as a solid precipitate.

Pharmaceutical granules provided by the present disclosure can comprise a crystalline 4-((L-valyl)oxy)butanoic acid salt such as crystalline 4-((L-valyl)oxy)butanoic acid hydrochloride or crystalline 4-((L-valyl)oxy)butanoic acid oxalate.

Pharmaceutical granules provided by the present disclosure can comprise immediate release pharmaceutical granules comprising a crystalline 4-((L-valyl)oxy)butanoic acid salt, modified release pharmaceutical granules comprising a crystalline 4-((L-valyl)oxy)butanoic acid salt, or a combination thereof.

Immediate release granules can comprise a plurality of immediate release granules.

Immediate release granules can comprise a homogeneous core comprising a crystalline 4-((L-valyl)oxy)butanoic acid salt and optionally an immediate release coating surrounding the core. Immediate release granules comprising an uncoated core are referred to as uncoated granules.

Uncoated immediate release granules can comprise, for example, greater than 70 wt % of a crystalline 4-((L-valyl)oxy)butanoic acid salt, where wt % is based on the total weight of the uncoated granules.

For example, uncoated immediate release pharmaceutical granules can comprise greater than 70 wt % of a crystalline 4-((L-valyl)oxy)butanoic acid salt, greater than 75 wt %, greater than 80 wt %, greater than 85 wt %, greater than 90 wt %, or greater than 95 wt % of a crystalline 4-((L-valyl)oxy)butanoic acid salt, where wt % is based on the total weight of the uncoated granules.

For example, uncoated immediate release pharmaceutical granules can comprise from 70 wt % to 98 wt % of a crystalline 4-((L-valyl)oxy)butanoic acid salt, from 75 wt % to 95 wt %, from 80 wt % to 95 wt %, from 85 wt % to 95 wt %, or from 87 wt % to 93 wt % of a crystalline 4-((L-valyl)oxy)butanoic acid salt, where wt % is based on the total weight of the uncoated granules.

An immediate release pharmaceutical granule can comprise, for example, a water-soluble polymer or a combination of water-soluble polymers.

Examples of suitable water-soluble polymers include hydroxypropyl cellulose, polyvinyl alcohol, hydroxypropylmethyl cellulose, hydroxypropylethyl cellulose, polyvinylpyrrolidone, polyethylene glycol, polyvinyl alcohol, povidone, crospovidone, and poloxamer.

A water-soluble polymer can comprise hydroxypropylmethyl cellulose.

An uncoated immediate release pharmaceutical granule can comprise, for example, from 1 wt % to 15 wt % of a water-soluble polymer, from 2 wt % to 13 wt %, from 3 wt % to 11 wt %, from 3 wt % to 9 wt %, from 4 wt % to 7 wt %, or from 4 wt % to 6 wt % of a water-soluble polymer such as hydroxypropylmethyl cellulose, where wt % is based on the total weight of the uncoated immediate release granule.

An uncoated immediate release pharmaceutical granule can comprise, for example, less than 15 wt % of a water-soluble polymer, less than 12 wt %, less than 10 wt %, less than 8 wt %, less than 6 wt %, less than 4 wt %, or less than 2 wt % of a water-soluble polymer, where wt % is based on the total weight of the uncoated immediate release granule.

An uncoated immediate release pharmaceutical granule can comprise, for example, greater than 1 wt % of a water-soluble polymer, greater than 3 wt %, greater than 5 wt %, greater than 7 wt %, greater than 9 wt %, greater than 11 wt %, or greater than 13 wt % of a water-soluble polymer, where wt % is based on the total weight of the uncoated immediate release granule.

An uncoated immediate release pharmaceutical granule can comprise, for example, an antistatic agent or a combination of antistatic agents.

Examples of suitable antistatic agents include talc (hydrous magnesium silicate), magnesium stearate, and silicon dioxide.

An antistatic agent can comprise hydrous magnesium silicate (talc).

An uncoated immediate release pharmaceutical granule can comprise, for example, from 1 wt % to 15 wt % of an antistatic agent, from 2 wt % to 13 wt %, from 3 wt % to 11 wt %, from 3 wt % to 9 wt %, from 4 wt % to 7 wt %, or from 4 wt % to 6 wt % of an antistatic agent such as talc, where wt % is based on the total weight of the uncoated immediate release granule.

An uncoated immediate release pharmaceutical granule can comprise, for example, less than 15 wt % of an antistatic agent, less than 12 wt %, less than 10 wt %, less than 8 wt %, less than 6 wt %, less than 4 wt %, or less than 2 wt % of an antistatic agent, where wt % is based on the total weight of the uncoated immediate release granule.

An uncoated immediate release pharmaceutical granule can comprise, for example, greater than 1 wt % of an antistatic agent, greater than 3 wt %, greater than 5 wt %, greater than 7 wt %, greater than 9 wt %, greater than 11 wt %, or greater than 13 wt % of an antistatic agent, where wt % is based on the total weight of the uncoated immediate release granule.

An uncoated immediate release pharmaceutical granule can comprise, for example, from 70 wt % to 98 wt % of a crystalline 4-((L-valyl)oxy)butanoic acid salt, from 1 wt % to 15 wt % of a water-soluble polymer such as hydroxypropylmethyl cellulose, and from 1 wt % to 15 wt % of an antistatic agent such as hydrous magnesium silicate, where wt % is based on the total weight of the uncoated immediate release pharmaceutical granule.

An uncoated immediate release pharmaceutical granule can comprise, for example, from 85 wt % to 95 wt % of a crystalline 4-((L-valyl)oxy)butanoic acid salt, from 2.5 wt % to 7.5 wt % of a water-soluble polymer such as hydroxypropylmethyl cellulose, and from 2.5 wt % to 7.5 wt % of an antistatic agent such as hydrous magnesium silicate, where wt % is based on the total weight of the uncoated immediate release pharmaceutical granule.

An uncoated immediate release pharmaceutical granule can comprise, for example, from 88 wt % to 92 wt % of a crystalline 4-((L-valyl)oxy)butanoic acid salt, from 4 wt % to 6 wt % of a water-soluble polymer such as hydroxypropylmethyl cellulose, and from 4 wt % to 6 wt % of an antistatic agent such as hydrous magnesium silicate, where wt % is based on the total weight of the uncoated immediate release pharmaceutical granule.

An uncoated immediate release pharmaceutical granule can comprise, for example, about 90 wt % of a crystalline 4-((L-valyl)oxy)butanoic acid salt, about 5 wt % of a water-soluble polymer such as hydroxypropylmethyl cellulose, and about 5 wt % of an antistatic agent such as hydrous magnesium silicate, where wt % is based on the total weight of the uncoated immediate release pharmaceutical granule.

Uncoated immediate release pharmaceutical granules can have an average granule size, for example, from 100 μm to 600 μm, from 150 μm to 550 μm, from 200 μm to 500 μm, from 250 μm to 450 μm or from 300 μm to 400 μm, where the average granule size is determined by sieve analysis or by laser diffraction.

Uncoated immediate release pharmaceutical granules can have an average granule size, for example, greater than 100 μm, greater than 200 μm, greater than 300 μm, greater than 400 μm, or greater than 500 μm, where the average granule size is determined by sieve analysis or by laser diffraction.

Uncoated immediate release pharmaceutical granules can have an average granule size, for example, of less than 600 μm, less than 500 μm, less than 400 μm, less than 300 μm, or less than 200 μm, where the average granule size is determined by sieve analysis or by laser diffraction.

Uncoated immediate release pharmaceutical granules can have a bulk density, for example, greater than 0.40 g/mL, greater than 0.50 g/mL, greater than 0.60 g/mL, greater than 0.90 g/mL, greater than 1.10 g/mL, greater than 1.30 g/mL, or greater than 1.50 g/mL, wherein bulk density is determined using a bulk density cylinder.

Uncoated immediate release pharmaceutical granules can have a bulk density, for example, from 0.40 g/mL to 1.60 g/mL, from 0.40 g/mL to 1.20 g/mL, from 0.40 g/mL to 0.80 g/mL, from 0.50 g/mL to 1.60 g/mL, from 0.50 g/mL to 1.40 g/mL, from 0.50 g/mL to 1.20 g/mL, from 0.60 g/mL to 1.60 g/mL, from 0.70 g/mL to 1.50 g/mL, from 0.80 g/mL to 1.40 g/mL, or from 1.00 g/mL to 1.20 g/mL, wherein bulk density is determined using a bulk density cylinder.

Uncoated immediate release pharmaceutical granules can have a bulk density, for example, from 0.60 g/mL to 1.60 g/mL, from 0.70 g/mL to 1.50 g/mL, from 0.80 g/mL to 1.40 g/mL, or from 1.00 g/mL to 1.20 g/mL, wherein bulk density is determined using a bulk density cylinder.

Uncoated immediate release pharmaceutical granules can have a bulk density, for example, from 0.5 g/mL to 1.0 g/mL, from 0.5 g/mL to 0.9 g/mL, from 0.5 g/mL to 0.8 g/mL, from 0.5 g/mL to 0.7 g/mL, or from 0.6 g/mL to 0.7 g/mL. An uncoated pharmaceutical granule can have a bulk density, for example, greater than 0.5 g/mL, greater than 0.6 g/mL, greater than 0.7 g/mL, greater than 0.8 g/mL, or greater than 0.9 g/mL, wherein bulk density is determined using a bulk density cylinder.

Uncoated immediate release pharmaceutical granules can be characterized by an average sphericity, for example, from 0.90 to 1, such as from 0.91 to 0.99, or from 0.92 to 0.98, where sphericity is determined using wet dispersion particle shape methods or by dynamic image analysis. Uncoated immediate release pharmaceutical granules provided by the present disclosure can be characterized by an average sphericity, for example, greater than 0.94, greater than 0.95, greater than 0.96, greater than 0.97, greater than 0.98, or greater than 0.99.

Uncoated immediate release pharmaceutical granules provided by the present disclosure can be characterized by a friability value, for example, from 0% to 2%. Granules with low friability are easier to coat than are granules with high friability. Granules with low friability are also more mechanically stable during storage and transportation. Friability is defined as the amount in wt % of granules having a diameter less than 75 μm that are generated by subjecting granules to a sonic sifter operated at a vibration amplitude of 8 corresponding to 3,600 sonic energy pulses per minute for at least 2 minutes. Uncoated immediate release pharmaceutical granules provided by the present disclosure can be characterized by a friability value, for example, of less than 2%, less than 1.8%, less than 1.6%, less than 1.4%, less than 1.2%, less than 1.0%, less than 0.8%, less than 0.6%, less than 0.4%, or less than 0.2%. Immediate release pharmaceutical granules provided by the present disclosure can have a friability, for example, of 1.02% where friability is determined using a sonic sifter.

Uncoated immediate release pharmaceutical granules can completely dissolve in water at a temperature from 20° C. to 25° C. such as 23° C. in less than 5 minutes following mixing and stirring in water, such as in less than 4 minutes, less than 3 minutes, less than 2 minutes, or in less than 1 minute. Immediate release pharmaceutical granules can at least partially dissolve in water at a temperature from 20° C. to 25° C. such as 23° C. in less than 5 minutes following mixing and stirring in water, such as in less than 4 minutes, less than 3 minutes, less than 2 minutes, or in less than 1 minute.

Uncoated immediate release pharmaceutical granules can be characterized by a dissolution profile in an aqueous buffer solution having a pH from 4.0 to 5.0 such as 4.5 at a temperature from 20° C. to 25° C. such as 23° C., in which 80 wt % of the granules are dissolved within 0.5 minutes, within 1 minute, within 2 minutes, within 3 minutes, within 4 minutes, or within 5 minutes.

Immediate release pharmaceutical granules can be a coated with a seal coating.

A seal coating can minimize the ingress of moisture into the pharmaceutically active ingredient and thereby increase the storage stability of the coated microparticle by reducing hydrolysis of the pharmaceutically active ingredient. A seal coating can also minimize negative interactions between the modified release coating and the pharmaceutically active ingredient, and thereby increase the storage stability of the coated immediate release granules by reducing hydrolysis of a crystalline 4-((L-valyl)oxy)butanoic acid salt. A seal coat is an immediate release coating.

A seal coating can comprise a water-soluble polymer such as, for example, hydroxypropylcellulose, polyvinyl alcohol, hydroxypropylmethyl cellulose, hydroxypropylethyl cellulose, polyvinylpyrrolidone, or polyethylene glycol.

A seal coating can have an average thickness, for example from 0.5 μm to 5 μm, from 1 μm to 4 μm, or from 1 μm to 3 μm. A seal coating can have an average thickness, for example, less than 5 μm, less than 4 μm, less than 3 μm, less than 2 μm, or less than 1 μm.

Coated immediate release pharmaceutical granules may not completely dissolve after mixing with water for longer than 1 minute, longer than 2 minutes, longer than 3 minutes, longer than 4 minutes, 5 minutes, or longer than 10 minutes.

Immediate release granules can comprise a plurality of coated or seal-coated granules having an immediate release functional coating. Immediate release granules comprising a plurality of coated granules can comprise greater than 80 wt % of a crystalline 4-((L-valyl)oxy)butanoic acid salt. Immediate release granules comprising coated granules can dissolve completely, for example, in less than 25 minutes, less than 20 minutes, less than 18 minutes, less than 16 minutes, less than 14 minutes, or less than 12 minutes, when tested in a USP Type 2 dissolution apparatus in a buffered solution at pH 4.5 at a temperature of 37° C. and a paddle speed of 100 rpm. Immediate release granules comprising coated granules can release greater than 80% of a crystalline 4-((L-valyl)oxy)butanoic acid salt, for example, in less than 10 minutes, less than 8 minutes, less than 6 minutes, or less than 4 minutes, when tested in a USP Type 2 dissolution apparatus in a buffered solution at pH 4.5 at a temperature of 37° C. and a paddle speed of 100 rpm. Coated immediate release granules can comprise a coating comprising a water-soluble-polymer such as, for example, hydroxypropylcellulose, polyvinyl alcohol, hydroxypropylmethyl cellulose, hydroxypropylethyl cellulose, polyvinylpyrrolidone, or polyethylene glycol. Coated immediate release granules can comprise a coating comprising an antistatic agent such as talc, magnesium stearate, or silicon dioxide.

Modified release granules can comprise a plurality of modified release pharmaceutical granules. Modified release pharmaceutical granules can comprise one or more modified release pharmaceutical granules where the different granules can have a different composition and/or property, such as for example, average granule size, active pharmaceutical ingredient, release profile, and/or excipient composition. Each of the two or more modified release pharmaceutical granules can have a different release profile in the gastrointestinal tract. Modified release pharmaceutical granules can comprise an uncoated or coated immediate release pharmaceutical granules with a modified release coating surrounding the uncoated immediate release pharmaceutical granules. Modified release pharmaceutical granules can comprise an uncoated immediate release pharmaceutical granule with a modified release coating surrounding the uncoated immediate release pharmaceutical granule.

Modified release granules can comprise a plurality of granules coated with a functional coating. A functional coating can comprise, for example, a modified release coating such as a controlled release coating, a sustained release coating, a pH-release coating, a pulsatile release coating, a timed-release coating, or a delayed release coating. A functional coating can be configured to release crystalline 4-((L-valyl)oxy)butanoic acid salt from a coated granule, for example, over an intended period of time following ingestion and/or in an intended region of the gastrointestinal tract.

Modified release granules can comprise uncoated granules comprising crystalline 4-((L-valyl)oxy)butanoic acid hydrochloride with one or more functional coatings surrounding the uncoated granule.

Each of the one or more functional coatings can independently have an average thickness, for example, of less than 50 μm, less than 40 μm, less than 30 μm, less than 20 μm, less than 15 μm, less than 10 μm, or less than 5 μm. Each of the one or more functional coatings can independently have an average thickness, for example, from 5 μm to 50 μm, from 5 μm to 40 μm, from 5 μm to 30 μm, from 5 μm to 20 μm, from 5 μm to 15 μm, or from 5 μm to 10 μm.

Modified release pharmaceutical granules provided by the present disclosure can comprising a plurality of modified release granules.

Modified release pharmaceutical granules can comprise, for example, from 60 wt % to 95 wt % of uncoated immediate release pharmaceutical granules, from 70 wt % to 90 wt %, from 76 wt % to 86 wt %, from 77 wt % to 85 wt %, from 78 wt % to 84 wt %, from 79 wt % to 83 wt %, from 70 wt % to 82 wt %, or about 81 wt % of uncoated immediate release pharmaceutical granules, where wt % is based on the total weight of the modified release pharmaceutical granules.

Modified release pharmaceutical granules can comprise, for example, greater than 60 wt % of immediate release pharmaceutical granules, greater than 65 wt %, greater than 70 wt %, greater than 75 wt %, greater than 80 wt %, greater than 85 wt %, greater than 90 wt % or greater than 95 wt % of uncoated immediate release pharmaceutical granules, where wt % is based on the total weight of the modified release pharmaceutical granules.

Modified release pharmaceutical granules can comprise, for example, 5 wt % to 40 wt %, of a modified release coating, from 10 wt % to 35 wt %, from 10 wt % to 30 wt %, from 14 wt % to 24 wt %, from 15 wt % to 23 wt %, from 16 wt % to 22 wt %, from 17 wt % to 21 wt %, from 18 wt % to 20 wt %, or about 19 wt % of a modified release coating, where wt % is based on the total weight of the modified release pharmaceutical granules.

Modified release pharmaceutical granules can comprise, for example, less than 40 wt % of a modified release coating, less than 30 wt %, less than 25 wt %, less than 20 wt %, less than 15 wt %, less than 10 wt % or less than 5 wt % of a modified release coating, where wt % is based on the total weight of the modified release pharmaceutical granules.

Modified release pharmaceutical granules can comprise, for example, from 60 wt % to 95 wt % of immediate release pharmaceutical granules and from 5 wt % to 40 wt % of a modified release coating, where wt % is based on the total weight of the modified release pharmaceutical granules.

Modified release pharmaceutical granules can comprise, for example, from 70 wt % to 90 wt % of immediate release pharmaceutical granules and from 10 wt % to 30 wt % of a modified release coating, where wt % is based on the total weight of the modified release pharmaceutical granules.

Modified release pharmaceutical granules can comprise, for example, from 75 wt % to 85 wt % of immediate release pharmaceutical granules and from 15 wt % to 25 wt % of a modified release coating, where wt % is based on the total weight of the modified release pharmaceutical granules.

Modified release pharmaceutical granules can comprise, for example, 77 wt % to 83 wt % of immediate release pharmaceutical granules and from 17 wt % to 23 wt % of a modified release coating, where wt % is based on the total weight of the modified release pharmaceutical granules.

Modified release pharmaceutical granules can comprise, for example, from 60 wt % to 90 wt % of a crystalline 4-((L-valyl)oxy)butanoic acid salt, from 65 wt % to 85 wt %, from 68 wt % to 78 wt %, from 69 wt % to 77 wt %, from 70 wt % to 76 wt %, from 71 wt % to 75 wt %, from 72 wt % to 74 wt %, or about 73 wt % of a crystalline 4-((L-valyl)oxy)butanoic acid salt, where wt % is based on the total weight of the modified release pharmaceutical granules.

Modified release pharmaceutical granules can comprise, for example, greater than 60 wt % of a crystalline 4-((L-valyl)oxy)butanoic acid salt, greater than 65 wt %, greater than 70 wt %, greater than 75 wt %, greater than 80 wt %, greater than 85 wt %, or greater than 90 wt % of a crystalline 4-((L-valyl)oxy)butanoic acid salt, where wt % is based on the total weight of the modified release pharmaceutical granules.

Modified release pharmaceutical granules can comprise, for example, less than 95 wt % of a crystalline 4-((L-valyl)oxy)butanoic acid salt, less than 90 wt %, less than 85 wt %, less than 80 wt %, less than 75 wt %, less than 70 wt % or less than 65 wt % of a crystalline 4-((L-valyl)oxy)butanoic acid salt, where wt % is based on the total weight of the modified release pharmaceutical granules.

A modified release coating can comprise a water-insoluble polymer, a water-soluble polymer, a lubricant, an antistatic agent, or a combination of any of the foregoing.

A modified release coating can comprise a water-insoluble polymer or a combination of water-insoluble polymers.

Examples of suitable water-insoluble polymers include ethylcellulose and polyvinyl acetates, polyacrylates, and polymethacrylates.

A water-insoluble polymer can comprise ethylcellulose.

A modified release coating can comprise, for example, from 60 wt % to 88 wt % of a water-insoluble polymer such as ethylcellulose, from 70 wt % to 78 wt %, from 69 wt % to 77 wt %, from 68 wt % to 76 wt %, or from 69 wt % to 75 wt % of a water-insoluble polymer such as ethylcellulose, where wt % is based on the total weight of the modified release coating.

A modified release coating can comprise a water-soluble polymer or a combination of water-soluble polymers.

Examples of suitable water-soluble polymers include hydroxypropylcellulose, polyvinyl alcohol, hydroxypropylmethyl cellulose, hydroxypropylethyl cellulose, polyvinylpyrrolidone, or polyethylene glycol.

A water-soluble polymer can comprise hydroxypropylcellulose.

A modified release coating can comprise, for example, from 1 wt % to 8 wt % of a water-soluble polymer such as hydroxypropylcellulose, from 2 wt % to 6 wt %, from 2.5 wt % to 5.5 wt %, from 3 wt % to 5.0 wt %, or from 3.5 wt % to 4.5 wt % of a water-soluble polymer such as hydroxypropylcellulose, where wt % is based on the total weight of the modified release coating.

A modified release coating can comprise a plasticizer or a combination of plasticizers.

Examples of suitable plasticizer include dibutyl sebacate, polyethylene glycol, triacetin, and triethyl citrate.

A plasticizer can comprise dibutyl sebacate.

A modified release coating can comprise, for example, from 2 wt % to 14 wt % of a plasticizer such as dibutyl sebacate, from 4 wt % to 12 wt %, from 5 wt % to 10 wt %, from 6 wt % to 9 wt %, or from 6 wt % to 8 wt % of a plasticizer such as dibutyl sebacate, where wt % is based on the total weight of the modified release coating.

A modified release coating can comprise an antistatic agent or a combination of antistatic agents.

Examples of suitable antistatic agents include talc (hydrous magnesium silicate), magnesium stearate, sodium stearyl fumarate, and silicon dioxide.

An antistatic agent can comprise hydrous magnesium silicate (talc).

A modified release coating can comprise, for example, from 5 wt % to 25 wt % of an antistatic agent such as hydrous magnesium silicate (talc), from 10 wt % to 20 wt %, from 11 wt % to 19 wt %, from 12 wt % to 18 wt %, 13 wt % to 17 wt %, or from 14 wt % to 16 wt %, of an antistatic agent such as hydrous magnesium silicate (talc), where wt % is based on the total weight of the modified release coating.

A modified release coating can comprise, for example, a water-insoluble polymer; a water-soluble polymer; a lubricant; and an antistatic agent.

A modified release coating can comprise from 60 wt % to 88 wt % of a water-insoluble polymer; from 2 wt % to wt % of a water-soluble polymer; from 6 wt % to 10 wt % of a lubricant; and from 10 wt % to 20 wt % wt % of an antistatic agent; wherein wt % is based on the total weight of the modified release coating.

A modified release coating can comprise from 63 wt % to 83 wt % of a water-insoluble polymer; from 1 wt % to 8 wt % of a water-soluble polymer; from 2 wt % to 14 wt % of a plasticizer, and from 5 wt % to 25 wt % wt % of an antistatic agent; wherein wt % is based on the total weight of the modified release coating.

In a modified release pharmaceutical granule or a modified release coating, the water-insoluble polymer can comprise ethylcellulose; the water-soluble polymer can comprise hydroxypropylcellulose; the lubricant can comprise dibutyl sebacate; and the antistatic agent can comprise hydrous magnesium silicate.

A modified release pharmaceutical granule can comprise, for example, from 11 wt % to 17 wt % of a water insoluble polymer such as ethylcellulose, from 12 wt % to 16 wt %, from 13 wt % to 15 wt %, or 14.14 wt % of a water insoluble polymer such as ethylcellulose, where wt % is based on the total weight of the modified release pharmaceutical granule.

A modified release pharmaceutical granule can comprise, for example, from 0.45 wt % to 1.05 wt % of a water-soluble polymer such as hydroxypropylcellulose, from 0.55 wt % to 0.95 wt %, from 0.65 wt % to 0.85 wt %, from 0.70 to 0.80 wt % or 0.75 wt % of a water-soluble polymer such as hydroxypropylcellulose, where wt % is based on the total weight of the modified release pharmaceutical granule.

A modified release pharmaceutical granule can comprise, for example, from 0.5 wt % to 2.5 wt % of a plasticizer such as dibutyl sebacate, from 0.75 wt % to 2.25 wt %, from 1.0 wt % to 2.0 wt %, or from 1.25 wt % to 1.75 wt % of a plasticizer such as dibutyl sebacate, where wt % is based on the total weight of the modified release pharmaceutical granule.

A modified release pharmaceutical granule can comprise, for example, from 1.0 wt % to 5.0 wt % of an antistatic agent such as hydrous magnesium silicate (talc), from 1.5 wt % to 4.5 wt %, from 2.0 wt % to 4.0 wt %, 2.5 wt % to 3.5 wt %, or from 2.8 wt % to 3.2 wt %, of an antistatic agent such as hydrous magnesium silicate (talc), where wt % is based on the total weight of the modified release pharmaceutical granule.

A modified release pharmaceutical granule can comprise, for example, from 70 wt % to 90 wt % of an uncoated immediate release pharmaceutical granule, from 11 wt % to 17 wt % of a water insoluble polymer such as ethylcellulose, from 0.45 wt % to 1.05 wt % of a water-soluble polymer such as hydroxypropylcellulose; from 0.5 wt % to 2.5 wt % of a plasticizer such as dibutyl sebacate; and from 1.0 wt % to 5.0 wt % of an antistatic agent such as hydrous magnesium silicate (talc), where wt % is based on the total weight of the modified release pharmaceutical granule.

A modified release coating can have a thickness, for example, from 5 μm to 40 μm, from 5 μm to 30 μm, from 5 μm to 20 μm, from 5 μm to 10 μm, from 10 μm to 40 μm, from 10 μm to 30 μm, or from 10 μm to 20 μm.

A modified release coating can have a thickness, for example, of less than 50 μm, less than 40 μm, less than 30, μm, less than 20 μm, or less than 10 μm.

A modified release pharmaceutical granule can comprise, for example, from 10 wt % to 45 wt % of the modified release coating, from 10 wt % to 40 wt %, or from 10 wt % to 30 wt %, where wt % is based on the total weight of the modified release pharmaceutical granule.

A modified release pharmaceutical granule can comprise, for example, of less than 45 wt %, less than 40 wt %, less than 35 wt %, less than 30 wt %, less than 25 wt %, less than 20 wt %, or less than 15 wt % of the modified release coating, where wt % is based on the total weight of the modified release pharmaceutical granule.

Modified release pharmaceutical granules can have an average granule size, for example, from 100 μm to 600 μm, from 150 μm to 550 μm, from 200 μm to 500 μm, from 250 μm to 450 μm, or from 300 μm to 400 μm, where the average granule size is determined by sieve analysis or laser diffraction.

Modified release pharmaceutical granules can have an average granule size, for example, greater than 100 μm, greater than 200 μm, greater than 300 μm, greater than 400 μm, or greater than 500 μm, where the average granule size is determined by sieve analysis or laser diffraction.

Modified release pharmaceutical granules can have an average granule size, for example, less than 600 μm, less than 550 μm, less than 500 μm, less than 450 μm, less, than 400 μm, less than 350 μm, less than 300 μm, or less than 250 μm, where the average granule size is determined by sieve analysis or laser diffraction.

Modified release pharmaceutical granules provided by the present disclosure can have an average a bulk density from 0.4 g/mL to 1.60 g/mL, 0.40 g/mL to 1.40 g/mL, 0.50 g/mL to 0.90 g/mL, from 0.55 g/mL to 0.80 g/mL, from 0.60 g/mL to 75 g/mL, or from 0.60 g/mL to 0.70 g/mL. Modified release pharmaceutical granules can have a bulk density, for example, greater than 0.50 g/mL, greater than 0.60 g/mL, greater than 0.80 g/mL, greater than 0.90 g/mL, greater than 1.00 g/mL, greater than 1.20 g/mL, or greater than 1.40 g/mL, where bulk density is determined using a bulk density cylinder. Modified release pharmaceutical granules can have a bulk density, for example, less than 1.6 g/mL, less than 1.20 g/mL, less than 1.00 g/mL, less than 0.80 g/mL, or less than 0.60 g/mL, where bulk density is determined using a bulk density cylinder.

Modified release pharmaceutical granules can have a water content, for example, less than 2 wt %, less than 1.5 wt % less than 1 wt %, less than 0.5 wt %, or less than 0.25 wt %, where wt % is based on the total weight of the modified release pharmaceutical granules.

Modified release pharmaceutical granules n can have a water content, for example, from 0.1 wt % to 2 wt %, from 0.1 wt % to 1 wt %, or from 0.2 wt % to 0.5 wt %, where wt % is based on the total weight of the modified release pharmaceutical granules.

Modified release pharmaceutical granules can be configured to release the crystalline 4-((L-valyl)oxy)butanoic acid hydrochloride continuously with time in the gastrointestinal tract.

Modified release pharmaceutical granules can have a dissolution profile in a buffered aqueous solution at a pH of 6.8 at a temperature of 23° C. characterized by from 20% to 40% dissolution at 1 hour, from 50% to 70% at 2 hours, from 60% to 80% at 3 hours, from 70% to 90% at 5 hours, and/or greater than 80% at 6 hours.

Modified release pharmaceutical granules can have a dissolution profile in a buffered aqueous solution at a pH of 6.8 at a temperature of 23° C. characterized by from 25% to 35% dissolution at 1 hour, from 55% to 65% at 2 hours, from 65% to 75% at 3 hours, from 75% to 85% at 5 hours, and/or greater than 80% at 6 hours.

A crystalline 4-((L-valyl)oxy)butanoic acid salt can be incorporated into a pharmaceutical composition to be administered to a patient by any appropriate route of administration including, for example, intradermal, intramuscular, intraperitoneal, intravenous, subcutaneous, intranasal, epidural, oral, peroral, sublingual, intracerebral, intravaginal, transdermal, rectal, inhalation, or topical. A pharmaceutical composition provided by the present disclosure can be an injectable formulation. A pharmaceutical composition provided by the present disclosure can be an injectable intravenous formulation. A pharmaceutical composition provided by the present disclosure can be an oral formulation. An oral formulation can be an oral dosage form.

Pharmaceutical compositions provided by the present disclosure can comprise a therapeutically effective amount of a crystalline 4-((L-valyl)oxy)butanoic acid salt together with a suitable amount of one or more pharmaceutically acceptable vehicles so as to provide a composition for proper administration to a patient. Suitable pharmaceutical vehicles and methods of preparing pharmaceutical compositions are described in the art.

A pharmaceutical composition provided by the present disclosure can provide a therapeutically effective amount of γ-hydroxybutyric acid in the systemic circulation of a patient following administration to the patient, such as following oral administration to the patient, and such as, for example from 6 to 12 hours following oral administration to the patient.

A crystalline 4-((L-valyl)oxy)butanoic acid salt and/or a pharmaceutical composition thereof can be used in an amount effective to achieve the intended purpose. For example, a crystalline 4-((L-valyl)oxy)butanoic acid salt or pharmaceutical composition thereof can be administered to a patient in a therapeutically effective amount to treat a disease or symptom of a disease such as a sleep disorder.

The amount of a crystalline 4-((L-valyl)oxy)butanoic acid salt and/or pharmaceutical composition thereof that will be effective in the treatment of a particular disorder or can depend in part on the nature of the disorder or condition, and can be determined by standard clinical techniques known in the art. In addition, in vitro or in vivo assays may optionally be employed to help identify optimal dosage ranges. A suitable amount of a crystalline 4-((L-valyl)oxy)butanoic acid salt and/or a pharmaceutical composition thereof administered can depend on, among other factors, the patient being treated, the weight of the patient, the severity of the affliction, the manner of administration and the judgment of the prescribing physician.

A crystalline 4-((L-valyl)oxy)butanoic acid salt can be assayed in vitro and in vivo, for the desired therapeutic activity, prior to use in humans. A crystalline 4-((L-valyl)oxy)butanoic acid salt can also be demonstrated to be effective and safe using animal model systems.

A therapeutically effective dose of a crystalline 4-((L-valyl)oxy)butanoic acid salt and/or pharmaceutical composition thereof can provide therapeutic benefit without causing substantial toxicity. Toxicity of a crystalline 4-((L-valyl)oxy)butanoic acid salt and/or a pharmaceutical composition thereof can be determined using standard pharmaceutical procedures and may be ascertained by one skilled in the art. The dose ratio between toxic and therapeutic effect is the therapeutic index. A crystalline 4-((L-valyl)oxy)butanoic acid salt and/or a pharmaceutical composition thereof can exhibit a high therapeutic index in treating a disease and disorder. A dose of a crystalline 4-((L-valyl)oxy)butanoic acid salt and/or pharmaceutical composition thereof can be within a range of circulating concentrations that include a therapeutically effective dose with minimal toxicity.

A pharmaceutical composition provided by the present disclosure can further comprise one or more pharmaceutically active compounds in addition to a crystalline 4-((L-valyl)oxy)butanoic acid salt. Such compounds can be provided to treat the same disease being treated with a crystalline 4-((L-valyl)oxy)butanoic acid salt or to treat a disease, disorder, or condition other than that being treated with a crystalline 4-((L-valyl)oxy)butanoic acid salt.

A crystalline 4-((L-valyl)oxy)butanoic acid salt or a pharmaceutical composition thereof can be used in combination with at least one other therapeutic agent. A crystalline 4-((L-valyl)oxy)butanoic acid salt or a pharmaceutical composition thereof may be administered to a patient together with another compound for treating a disease such as a sleep disorder in the patient. A crystalline 4-((L-valyl)oxy)butanoic acid salt and the at least one other therapeutic agent can act additively or synergistically. The at least one additional therapeutic agent can be included in the same pharmaceutical composition comprising a crystalline 4-((L-valyl)oxy)butanoic acid salt or can be in a separate pharmaceutical composition. Accordingly, methods provided by the present disclosure include, in addition to administering a crystalline 4-((L-valyl)oxy)butanoic acid salt, administering one or more therapeutic agents effective for treating a different disease, disorder or condition other than the disease being treated with a crystalline 4-((L-valyl)oxy)butanoic acid salt. Methods provided by the present disclosure include administering crystalline 4-((L-valyl)oxy)butanoic acid salt or a pharmaceutical composition thereof and one or more other therapeutic agents provided that the combined administration does not inhibit the therapeutic efficacy of a crystalline 4-((L-valyl)oxy)butanoic acid salt and/or γ-hydroxybutyric acid and/or does not produce adverse combination effects.

A pharmaceutical composition comprising a crystalline 4-((L-valyl)oxy)butanoic acid salt can be administered concurrently with the administration of another therapeutic agent, which may be part of the same pharmaceutical composition as, or in a different pharmaceutical composition than that comprising a crystalline 4-((L-valyl)oxy)butanoic acid salt. A crystalline 4-((L-valyl)oxy)butanoic acid salt or a pharmaceutical composition thereof may be administered prior or subsequent to administration of another therapeutic agent. In certain embodiments of combination therapy, the combination therapy may comprise alternating between administering a crystalline 4-((L-valyl)oxy)butanoic acid salt and a pharmaceutical composition comprising another therapeutic agent such as to minimize adverse drug effects associated with a particular drug. When a crystalline 4-((L-valyl)oxy)butanoic acid salt is administered concurrently with another therapeutic agent that potentially may produce an adverse drug effect including, for example, toxicity, the other therapeutic agent may be administered at a dose that falls below the threshold at which the adverse drug reaction is elicited.

A pharmaceutical composition comprising a crystalline 4-((L-valyl)oxy)butanoic acid salt may be administered with one or more substances, for example, to enhance, modulate and/or control release, bioavailability, therapeutic efficacy, therapeutic potency, and/or stability of a crystalline 4-((L-valyl)oxy)butanoic acid salt. For example, to enhance the therapeutic efficacy of a crystalline 4-((L-valyl)oxy)butanoic acid salt or a pharmaceutical composition comprising a crystalline 4-((L-valyl)oxy)butanoic acid salt may be co-administered with one or more active agents to increase the absorption or diffusion and/or transport of 4-((L-valyl)oxy)butanoic acid salt from the gastrointestinal tract into the systemic circulation, or to inhibit degradation of a crystalline 4-((L-valyl)oxy)butanoic acid salt in the blood of a patient. A pharmaceutical composition comprising a crystalline 4-((L-valyl)oxy)butanoic acid salt may be co-administered with an active agent having pharmacological effects that enhance the therapeutic effectiveness of a crystalline 4-((L-valyl)oxy)butanoic acid salt or the therapeutic efficacy of γ-hydroxybutyric acid.

For oral therapeutic administration, a crystalline 4-((L-valyl)oxy)butanoic acid salt can be incorporated with excipients and used, for example, in the form of tablets, buccal tablets or tabs, troches, capsules, elixirs, suspensions, syrups, or wafers, to be admixed with an aqueous medium. A dosage form can comprise a therapeutically effective amount of a crystalline 4-((L-valyl)oxy)butanoic acid salt or less than a therapeutically effective amount of a crystalline 4-((L-valyl)oxy)butanoic acid salt.

A pharmaceutical composition or oral dosage form can comprise, for example, from 0.5 gram to 30 grams such as from 1 gram to 20 grams of a crystalline 4-((L-valyl)oxy)butanoic acid salt.

An oral formulation such as an oral dosage form can contain, for example, from 0.5 gram-equivalents to 15 gram-equivalents such as from 1 gram-equivalents to 10 gram-equivalents of γ-hydroxybutyric acid.

An oral dosage form provided by the present disclosure can be in the form, for example, of a tablet, a troche, a pill, a capsule, a dispersion, a suspension, a solution, or a plurality of granules.

An oral dosage form can comprise an immediate release component and a modified release component.

A pharmaceutical composition or oral dosage form can comprise pharmaceutical granules, wherein the pharmaceutical granules comprise a crystalline 4-((L-valyl)oxy)butanoic acid salt. The granules can comprise an immediate release granulation comprising a crystalline 4-((L-valyl)oxy)butanoic acid salt, modified release granules comprising a crystalline 4-((L-valyl)oxy)butanoic acid salt, or a combination thereof.

A pharmaceutical composition can comprise, for example, greater than 1 wt % of immediate release granules, greater than 5 wt %, greater than 10 wt %, greater than 20 wt %, greater than 30 wt %, greater than 40 wt %, greater than 50 wt %, greater than 60 wt %, greater than 70 wt %, greater than 80 wt %, or greater than 90 wt % of immediate release granules, where wt % is based on the total weight of the pharmaceutical composition.

A pharmaceutical composition can comprise, for example, less than 99 wt % of immediate release granules, less than 95 wt %, less than 90 wt %, less than 80 wt %, less than 70 wt %, less than 60 wt %, less than 50 wt %, less than 40 wt %, less than 30 wt %, less than 20 wt % or less than 10 wt % of immediate release granules, where wt % is based on the total weight of the pharmaceutical composition.

A pharmaceutical composition can comprise, for example, from 1 wt % to 99 wt % of immediate release granules, from 5 wt % to 95 wt %, from 10 wt % to 80 wt %, from 10 wt % to 70 wt %, from 15 wt % to 60 wt %, from 15 wt % to 50 wt %, from 20 wt % to 40 wt %, or from 20 wt % to 30 wt %, where wt % is based on the total weight of the pharmaceutical composition.

A pharmaceutical composition can comprise, for example, greater than 1 wt % of modified release granules, greater than 5 wt %, greater than 10 wt %, greater than 20 wt %, greater than 30 wt %, greater than 40 wt %, greater than 50 wt %, greater than 60 wt %, greater than 70 wt %, greater than 80 wt %, or greater than 90 wt % of modified release granules, where wt % is based on the total weight of the pharmaceutical composition.

A pharmaceutical composition can comprise, for example, less than 99 wt % of modified release granules, less than 95 wt %, less than 90 wt %, less than 80 wt %, less than 70 wt %, less than 60 wt %, less than 50 wt %, less than 40 wt %, less than 30 wt %, less than 20 wt % or less than 10 wt % of modified release granules, where wt % is based on the total weight of the pharmaceutical composition.

A pharmaceutical composition can comprise, for example, from 1 wt % to 99 wt % of modified release granules, from 10 wt % to 95 wt %, from 20 wt % to 90 wt %, from 30 wt % to 90 wt %, from 40 wt % to 85 wt %, from 50 wt % to 85 wt %, from 55 wt % to 80 wt %, or from 60 wt % to 60 wt % of modified release granules, where wt % is based on the total weight of the pharmaceutical composition.

Pharmaceutical granules can comprise, for example, from 17 wt % to 37 wt % of immediate release pharmaceutical granules; and from 63 wt % to 83 wt % of modified release pharmaceutical granules, wherein wt % is based on the total weight of the pharmaceutical granules.

In a combination of pharmaceutical granules, immediate release pharmaceutical granules can comprise, for example, from 0.1 g to 2 g such as from 0.1 g to 10 g of a crystalline 4-((L-valyl)oxy)butanoic acid salt; and modified release pharmaceutical granules can comprise from 0.1 g to 20 g of a crystalline 4-((L-valyl)oxy)butanoic acid salt.

In a combination of pharmaceutical granules, immediate release pharmaceutical granules can comprise, for example, from 2.5 g to 6.5 g of a crystalline 4-((L-valyl)oxy)butanoic acid salt; and modified release pharmaceutical granules can comprise from 8 g to 12 g of a crystalline 4-((L-valyl)oxy)butanoic acid salt.

An oral dosage form provided by the present disclosure can comprise, for example, from 0.1 grams to 20 grams of a crystalline 4-((L-valyl)oxy)butanoic acid salt, from 0.1 grams to 15 grams, from 0.1 grams to 12 grams, from 0.1 grams to 10 grams, from 0.2 grams to 8 grams, from 0.5 grams to 5 grams, from 1 gram to 4.5 grams, or from 1.5 grams to 4 grams of a crystalline 4-((L-valyl)oxy)butanoic acid salt. An oral dosage form can comprise, for example, greater than 0.1 grams, greater than 0.5 grams, greater than 1 gram, greater than 2 grams, greater than 3 grams, greater than 4 grams, greater than 6 grams, or greater than 8 grams greater than 10 grams, greater than 14 grams, or greater than 18 grams of a crystalline 4-((L-valyl)oxy)butanoic acid salt. An oral dosage form can comprise, for example, less than 18 grams of a crystalline 4-((L-valyl)oxy)butanoic acid salt, less than 16 grams, less than 14 grams, less than 12 grams, less than 10 grams, less than 8 grams, less than 6 grams, less than 4 grams, or less than 2 grams of a crystalline 4-((L-valyl)oxy)butanoic acid salt.

An oral dosage form can comprise an oral suspension of coated granules having a modified release functional coating. An oral dosage form can comprise modified release granules.

An oral dosage form can comprise a combination of immediate release granules and modified release granules.

An oral dosage form can comprise a suspension of modified release granules suspended in an aqueous solution comprising a dissolved crystalline 4-((L-valyl)oxy)butanoic acid salt and partially dissolved immediate release granules.

An oral dosage form can comprise a suspension of modified release granules suspended in an aqueous solution comprising a dissolved crystalline 4-((L-valyl)oxy)butanoic acid salt.

An oral dosage form provided by the present disclosure can provide a therapeutically effective amount of 4-((L-valyl)oxy)butanoic acid or a 4-((L-valyl)oxy)butanoic acid salt in the systemic circulation of a patient over a period of time.

For example, an oral dosage form provided by the present disclosure can provide a therapeutically effective amount of 4-((L-valyl)oxy)butanoic acid in the systemic circulation of a patient over a period of 3 hours, 6, hours 8, hours, or 10 hours following oral administration.

An oral dosage form provided by the present disclosure can provide a therapeutically effective amount of a 4-((L-valyl)oxy)butanoic acid salt over a period from 4 hours to 12 hours, from 4 hours to 10 hours, from 4 hours to 8 hours, or from 4 hours to 6 hours following oral administration.

An oral dosage form provided by the present disclosure can provide a therapeutically effective amount of 4-((L-valyl)oxy)butanoic acid over a duration of from 1 hour to 12 hours following oral administration, from 2 hours to 10 hours, or from 4 hours to 8 hours following oral administration.

An oral dosage form provided by the present disclosure can be a once nightly oral dosage form. For a once nightly oral dosage form, a patient can administer a dose of a crystalline 4-((L-valyl)oxy)butanoic acid salt before going to bed and sleep through the night such as for 6 hours or for 8 hours without having to administer a second dose during the night.

An oral dosage form provided by the present disclosure can provide a therapeutically effective amount of a γ-hydroxybutyric acid in the plasma of a patient.

An oral dosage form provided by the present disclosure can provide a therapeutically effective amount of γ-hydroxybutyric acid in the plasma of a patient for a period of 4 hours, 6, hours, 8 hours, or 10 hours following oral administration of the modified release oral composition.

An oral dosage form provided by the present disclosure can provide a plasma concentration of γ-hydroxybutyric acid greater than 10 μg/mL for more than 4 hours, more than 6 hours, more than 8 hours, or more than 10 hours following oral administration of the modified release oral composition.

Pharmaceutical granules provided by the present disclosure can have an average bulk density, for example, from 0.6 g/mL to 0.9 g/mL, from 0.65 g/mL to 0.85 g/mL, or from 0.7 g/mL to 0.8 g/mL, where the average granule bulk density is determined using a bulk density cylinder.

Pharmaceutical granules provided by the present disclosure can have an average tapped density that is, for example, within ±20% of the average tapped density of excipient granules with which the pharmaceutical granules are combined, within ±15%, within ±10%, or within ±5% of the average tapped density of excipient granules with which the pharmaceutical granules are combined, where average granule tapped density is determined according to USP <616>.

Pharmaceutical granules provided by the present disclosure can have an average tapped density, for example, from 500 g/mL to 900 g/mL, from 550 g/L to 850 g/mL, from 600 g/mL to 800 g/mL, or from 650 g/mL to 750 g/mL, where the average granule tapped density is determined according to USP <616>.

Pharmaceutical granules provided by the present disclosure can have a Hausner ratio that is, for example, within ±20% of the average Hausner ratio of excipient granules with which the pharmaceutical granules are combined, within ±15%, within ±10%, or within ±5% of the Hausner ratio of an excipient with which the pharmaceutical granules are combined, where the Hausner ratio is determined according to USP <1174>.

Pharmaceutical granules provided by the present disclosure can have a Hausner ratio, for example, from 1.0 to 1.5, from 1.1 to 1.4, from 1.1 to 1.35, or from 1.1 to 1.25, where the Hausner ratio is determined according to USP <1174>.

Pharmaceutical granules provided by the present disclosure can have a compressibility index that is, for example, within ±20% of the compressibility index of excipient granules with which the pharmaceutical granules are combined, within ±15%, within ±10%, or within ±5% of the compressibility index of excipient granules with which the pharmaceutical granules is combined.

Pharmaceutical granules provided by the present disclosure can have a compressibility index, for example, from 8 to 22, from 10 to 20, or from 12 to 18.

Pharmaceutical granules provided by the present disclosure can have a Flodex® value that is, for example, within ±20% of the Flodex® value of excipient granules with which the pharmaceutical granules are combined, within ±15%, within ±10%, or within ±5% of the Flodex® value of a excipient granules with which the pharmaceutical granules are combined, where the Flodex® value is determined according to USP <1174>.

Pharmaceutical granules provided by the present disclosure can have a Flodex® value, for example, of less than 5 mm, less than 4 mm, less than 3 mm, or less than 2 mm, where the Flodex® value is determined according to USP <1174>.

Pharmaceutical granules provided by the present disclosure can have a friability value, for example, of less than 0.2 such as about 0.1 where friability is determined using an ionic sifter.

A pharmaceutical composition provided by the present disclosure can comprise excipient granules or a combination of excipient granules. Examples of suitable excipient granules are disclosed in U.S. Application Publication No. 2025/0041425 A1 filed on Jul. 31, 2024, and in U.S. Application Publication No. 2025/0041258 A1 filed on Jul. 31, 2024, each of which is incorporated by reference in its entirety.

Excipient granules provided by the present disclosure comprises a plurality of excipient granules. Excipient granules provided by the present disclosure can comprise pharmaceutically acceptable excipients.

When combined with water, excipient granules provides a viscous aqueous solution. The viscosity of the viscous aqueous solution can be selected to improve the palatability of a pharmaceutical composition comprising pharmaceutical granules dispersed and suspended in the viscous solution. A suitable viscosity effective in improving the palatability of pharmaceutical granules can depend, for example, on the average size of the pharmaceutical granules, the size distribution of the pharmaceutical granules, the density of the pharmaceutical granules, the volume percent of the pharmaceutical granules in the viscous aqueous solution, or a combination of any of the foregoing. In general, it is desirable that the viscosity of an oral pharmaceutical composition for administration to a patient be drinkable. A drinkable composition can have a viscosity, for example, similar to that of a drinkable yogurt. For example, a drinkable composition can have viscosity from 50 cp to 600 cP, determined using a No. 62 spindle at a speed of 12 rpm according to USP <912>, Method 1C.

Excipient granules provided by the present disclosure can comprise a viscosifying agent, a disintegrant, and one or more additional excipients.

An excipient granule provided by the present disclosure can comprise an amount of each of the excipients suitable to accomplish an intended purpose of excipient granules such as, for example, to improve the palatability of pharmaceutical granules.

Excipient granules provided by the present disclosure can provide a viscous solution within 90 seconds, such as within 30 seconds of being combined and mixed with an aqueous solution at a temperature from 20° C. to 25° C., such as 23° C. The maximum viscosity of the aqueous solution can be, for example, from 10 cP to 200 cP, from 25 CP to 150 cP, from 50 cP to 100 cP, from 40 cP to 80 cP, or from 50 cP to 70 cP at a temperature from 20° C. to 25° C., such as 23 GC.

When combined with an aqueous solution excipient granules can provide a viscous solution comprising pharmaceutical granules dispersed and suspended in the viscous solution that are palatable for oral ingestion. The additional excipients of excipient granules provided by the present disclosure can be selected, for example, to reduce the sensation of granularity of the pharmaceutical granules and to enhance the taste of the pharmaceutical composition. The additional excipients can be selected to adjust the pH of the pharmaceutical composition and to enhance the storage stability of the excipient granules and/or pharmaceutical granules.

Excipient granules provided by the present disclosure can comprise, for example, a viscosifying agent, a disintegrant, and one or more additional excipients. The one or more additional excipients is in addition to the viscosifying agent and the disintegrant.

Excipient granules provided by the present disclosure can improve the palatability of orally administered pharmaceutical granules.

Palatability can be defined as the overall appreciation of an oral medicinal product in relation to its smell, taste, aftertaste, and texture or feeling in the mouth. Palatability includes the overall acceptance of the taste, flavor, smell, does volume or size and texture of a medicine to be administered to the mouth and/or to be swallowed. excipient granules provided by the present disclosure can increase the viscosity of an orally administered solution comprising pharmaceutical granules and thereby reduce the coarse or rough feeling in the mouth.

To enhance the convenience during use, an excipient granulation can provide a viscous solution capable of suspending pharmaceutical granules within 30 seconds after being combined with water.

A suitable solution viscosity can be determined by a number of factors such as the density of the pharmaceutical granules, the average size of the granules, the size distribution of the granules, and the average volume of the granules.

Excipient granules provided by the present disclosure can be particularly useful with high dose drugs such as γ-hydroxybutyric acid. High dose drugs can require that large tablets or multiple tablets be administered. The inconvenience and/or discomfort of administering high dose drugs in the form of tablets can be avoided by using pharmaceutical granules. However, high dose granules can produce a coarse or rough sensation in the mouth.

For convenience, it is desirable that a viscous solution prepared using excipient granules provided by the present disclosure be drinkable. A drinkable solution can have a viscosity, for example, from 50 cP to 500 cP at a temperature from 20° C. to 25° C., such as 23° C., determined using a No. 62 spindle at a speed of 12 rpm according to USP <912>, Method 1C. excipient granules provided by the present disclosure can be configured to provide a viscosity, when mixed with 40 mL of water at 23° C., for example, from 20 cP to 100 cP, determined using a No. 62 spindle at a speed of 12 rpm according to USP <912>, Method 1C.

However, excipient granules can also be used to provide higher viscosity pharmaceutical compositions that are more appropriately be eaten. For example, a pharmaceutical composition can have a viscosity from 100 cP to 500 cP at a temperature from 20° C. to 25° C., such as 23° C., determined using a No. 62 spindle at a speed of 12 rpm according to USP <912>, Method 1C.

Intended objectives of excipient granules provided by the present disclosure include, for example, (1) to provide a viscous solution having a viscosity from 10 cP to 200 cP such as from 40 cP to 80 cP, or from 50 cP to 70 cP, following mixing with an aqueous solution at 23° C. in less than 90 seconds such as in less than 30 seconds; (2) to provide an average excipient granule size similar to that of pharmaceutical granules; (3) to provide excipient granules that are physically robust to storage and transportation; and (4) to provide a homogeneous granule composition such that the excipient granules and other granules such as modified release pharmaceutical granules in the composition do not segregate and remain homogeneously dispersed throughout the suspension. It is desirable that the excipient granules and other granules in the composition remain homogeneously dispersed to facilitate the ability of the reconstituted solution and/or suspension to be homogeneous. Additional intended objectives can include (1) taste enhancement; (2) pH maintenance; (3) rapid dissolution, and (4) low wt % or low volume.

In an oral pharmaceutical composition provided by the present disclosure, the oral pharmaceutical composition can have a viscosity, for example, from 60 cP to 600 cP, such as from 200 cP to 600 cP, of from 300 cP to 600 cP, after 30 seconds following mixing with 40 mL of water at 23° C., determined using a No. 62 spindle at a speed of 12 rpm according to USP <912>, Method 1C. In an oral pharmaceutical composition provided by the present disclosure, the oral pharmaceutical composition can have a viscosity, for example, from 200 cP to 1,100 cP, such as from 400 cP to 1,000 cP, or from 600 cP to 1,000 cP, after 60 seconds following mixing with 40 mL of water at 23° C., determined using a No. 62 spindle at a speed of 12 rpm according to USP <912>, Method 1C. An oral pharmaceutical composition can have a viscosity similar to the viscosity of a drinkable yogurt product.

In an oral pharmaceutical composition provided by the present disclosure, the oral pharmaceutical composition can have a viscosity, for example, from 60 cP to 600 cP, such as from 200 cP to 600 cP, of from 300 cP to 600 cP, from 15 seconds to 45 seconds following mixing with 40 mL of water at 23° C., determined using a No. 62 spindle at a speed of 12 rpm according to USP <912>, Method 1C. In an oral pharmaceutical composition provided by the present disclosure, the oral pharmaceutical composition can have a viscosity, for example, from 200 cP to 1,100 cP, such as from 400 cP to 1,000 cP, or from 600 cP to 1,000 cP, from 45 seconds to 75 seconds following mixing with 40 mL of water at 23° C., determined using a No. 62 spindle at a speed of 12 rpm according to USP <912>, Method 1C. An oral pharmaceutical composition can have a viscosity similar to the viscosity of a drinkable yogurt product.

In addition to excipient granules, the viscosity of an oral pharmaceutical formulation will be determined by the dissolution of the immediate release pharmaceutical granules, and by the suspended modified release pharmaceutical granules.

A pharmaceutical composition provided by the present disclosure can comprise immediate release pharmaceutical granules and a modified release pharmaceutical granules.

A pharmaceutical composition can comprise, for example, from 20 wt % to 30 wt % of immediate release pharmaceutical granules, from 22 wt % to 28 wt %, or from 24 wt % to 26 wt % of immediate release pharmaceutical granules, where wt % is based on the total weight of the pharmaceutical composition.

A pharmaceutical composition can comprise, for example, from 62 wt % to 72 wt % of immediate release pharmaceutical granules, from 64 wt % to 70 wt %, or from 66 wt % to 68 wt % of immediate release pharmaceutical granules, where wt % is based on the total weight of the pharmaceutical composition.

A pharmaceutical composition can comprise, for example, from 2 wt % to 12 wt % of excipient granules, from 4 wt % to 10 wt % or from 6 wt % to 8 wt % of excipient granules, where wt % is based on the total weight of the pharmaceutical composition.

A pharmaceutical composition can comprise, for example, from 2.5 wt % to 12.5 wt % of the excipient granules, from 3.5 wt % to 11.5 wt %, from 4.5 wt % to 10.5 wt %, from 5.5 wt % to 9.5 wt %, from 6.5 wt % to 8.5 wt %, from 7.0 wt % to 8.0 wt %, or 7.5 wt %, where wt % is based on the total weight of the pharmaceutical composition.

A pharmaceutical composition can comprise, for example, from 20 wt % to 30 wt % of the immediate release pharmaceutical granules n, from 62 wt % to 72 wt % of the modified release pharmaceutical granules, and from 2 wt % to 12 wt % of the excipient granules, where wt % is based on the total weight of the pharmaceutical composition.

A pharmaceutical composition can comprise, for example, from 22 wt % to 28 wt % of the immediate release pharmaceutical granules, from 64 wt % to 72 wt % of the modified release pharmaceutical granules, and from 4 wt % to 10 wt % of the excipient granules, where wt % is based on the total weight of the pharmaceutical composition.

A pharmaceutical composition can comprise, for example, from 24 wt % to 26 wt % of the immediate release pharmaceutical granules, from 66 wt % to 70 wt % of the modified release pharmaceutical granules, and from 6 wt % to 9 wt % of the excipient granules, where wt % is based on the total weight of the pharmaceutical composition.

The pharmaceutical composition can comprise, for example, from 24 wt % to 38 wt % of the crystalline 4-((L-valyl)oxy)butanoic acid salt in the immediate release component and from 62 wt % to 76 wt % of the crystalline 4-((L-valyl)oxy)butanoic acid salt in the modified release component, where wt % is based on the total weight of the crystalline 4-((L-valyl)oxy)butanoic acid salt in the pharmaceutical composition.

The pharmaceutical composition can comprise, for example, from 26 wt % to 36 wt % of the crystalline 4-((L-valyl)oxy)butanoic acid salt in the immediate release component and from 64 wt % to 74 wt % of the crystalline 4-((L-valyl)oxy)butanoic acid salt in the modified release component, where wt % is based on the total weight of the crystalline 4-((L-valyl)oxy)butanoic acid salt in the pharmaceutical composition.

The pharmaceutical composition can comprise, for example, from 28 wt % to 34 wt % of the crystalline 4-((L-valyl)oxy)butanoic acid salt in the immediate release component and from 66 wt % to 72 wt % of the crystalline 4-((L-valyl)oxy)butanoic acid salt in the modified release component, where wt % is based on the total weight of the crystalline 4-((L-valyl)oxy)butanoic acid salt in the pharmaceutical composition.

The pharmaceutical composition can comprise, for example, from 30 wt % to 32 wt % of the crystalline 4-((L-valyl)oxy)butanoic acid salt in the immediate release component and from 68 wt % to 70 wt % of the crystalline 4-((L-valyl)oxy)butanoic acid salt in the modified release component, where wt % is based on the total weight of the crystalline 4-((L-valyl)oxy)butanoic acid salt in the pharmaceutical composition.

In a pharmaceutical composition, the ratio of the weight of a crystalline 4-((L-valyl)oxy)butanoic acid salt in the modified release pharmaceutical granules to the weight of a crystalline 4-((L-valyl)oxy)butanoic acid salt in the immediate release pharmaceutical granules can be, for example, from 1.7 to 3.7, from 1.9 to 3.5, from 2.1 to 3.3, from 2.3 to 3.1, or from 2.5 to 2.9.

In a pharmaceutical composition, the modified release pharmaceutical granules can comprise, for example, from 64 wt % to 74 wt % of the a crystalline 4-((L-valyl)oxy)butanoic acid salt and the immediate release pharmaceutical granules can comprise from 26 wt % to 36 wt % of the crystalline 4-((L-valyl)oxy)butanoic acid salt, wherein wt % is based on the total weight of the crystalline 4-((L-valyl)oxy)butanoic acid salt in the pharmaceutical composition.

A pharmaceutical composition and an oral pharmaceutical composition provided by the present disclosure can comprise a therapeutically effective dose of a crystalline 4-((L-valyl)oxy)butanoic acid salt.

For a 14.5 g dose of a crystalline 4-((L-valyl)oxy)butanoic acid salt, 10 g of crystalline 4-((L-valyl)oxy)butanoic acid salt can be in the modified release pharmaceutical granules, and 4.5 g of a crystalline 4-((L-valyl)oxy)butanoic acid salt can be in the immediate release pharmaceutical granules.

For a 14.5 g dose of a crystalline 4-((L-valyl)oxy)butanoic acid salt, from 8 g to 12 g of crystalline 4-((L-valyl)oxy)butanoic acid salt can be in the modified release pharmaceutical granules, and 2.5 g to 6.5 g of a crystalline 4-((L-valyl)oxy)butanoic acid salt can be in the immediate release pharmaceutical granules.

A 14.5 g dose of a crystalline 4-((L-valyl)oxy)butanoic acid salt can comprise 13.34 g of the modified release pharmaceutical granules, 5 g of the immediate release pharmaceutical granules, 1.50 g of the excipient granules, and 0.10 g of an antistatic agent such as talc.

A 14.5 g dose of a crystalline 4-((L-valyl)oxy)butanoic acid salt can comprise from 11.34 g to 15.35 g of the modified release pharmaceutical granules, 3 g to 7 g of the immediate release pharmaceutical granules, from 1 g to 2 g of the excipient granules, and from 0.05 g to 0.15 g of an antistatic agent such as talc.

A dose of a crystalline 4-((L-valyl)oxy)butanoic acid salt can comprise, for example, from 1 g to 30 g of a crystalline 4-((L-valyl)oxy)butanoic acid salt, such as from 2 g to 25 g, from 4 g to 20 g, from 5 g to 20 g, or from 10 g to 15 g of a crystalline 4-((L-valyl)oxy)butanoic acid salt.

A pharmaceutical composition can comprise a unit dose of a crystalline 4-((L-valyl)oxy)butanoic acid salt, where a unit dose refers to a single dose that is capable of treating an intended disease or symptom of a disease.

An oral pharmaceutical composition provided by the present disclosure can comprise a viscous aqueous solution and an active pharmaceutical ingredient. An oral pharmaceutical composition can be formed by combining a pharmaceutical composition with an aqueous solution such as water. An oral pharmaceutical composition orally ingestible by a patient.

An oral pharmaceutical composition provided by the present disclosure can comprise an oral pharmaceutical composition for oral ingestion prepared by mixing a pharmaceutical composition provided by the present disclosure with water or an aqueous solution to provide a viscous solution comprising modified release pharmaceutical granules provided by the present disclosure dispersed and suspended in the viscous solution. The viscous solution can comprise the constituents of the excipient granules dissolved in water or the aqueous solution. The viscous solution can also comprise a crystalline 4-((L-valyl)oxy)butanoic acid salt dissolved in the viscous solution where the comprise a crystalline 4-((L-valyl)oxy)butanoic acid salt is provided by the partially or fully dissolved immediate release pharmaceutical granules. The viscous solution can further comprise a partially dissolved immediate release pharmaceutical granules suspended and dispersed in the viscous solution.

An oral pharmaceutical composition provided by the present disclosure can comprise a viscous aqueous solution. The oral pharmaceutical composition is intended for oral administration.

A viscous aqueous solution can be prepared by combining and mixing an excipient granules provided by the present disclosure with an aqueous solution.

When excipient granules are mixed with water, the contents of the excipient granules provide a concentration, for example, from 0.005 g/mL to 0.060 g/mL of the excipient granules, from 0.010 g/mL to 0.055 g/mL, from 0.015 g/mL to 0.050 g/mL, from 0.015 g/mL to 0.045 g/mL, from 0.02 g/mL to 0.04 g/mL or from 0.025 g/mL to 0.035 g/mL of the excipient granules, where g/mL is based on the grams of the excipient granules mixed with the volume of water.

For example, an oral pharmaceutical composition can be prepared, for example, by mixing from 0.2 g to 2.2 g of the excipient granules in 40 mL of water, from 0.4 g to 2.0 g, from 0.6 g to 1.8 g, from 0.8 g to 1.6 g, from 1.0 g to 1.4 g of the excipient granules in 40 mL of water.

An oral pharmaceutical composition provided by the present disclosure can be prepared by combining excipient granules and pharmaceutical granules with water. The excipient granules and the pharmaceutical granules can be combined with water either simultaneously or sequentially. For example, excipient granules can first be combined with water to form a viscous solution, and pharmaceutical granules subsequently combined and mixed with the viscous solution. Alternatively, pharmaceutical granules can first be combined with water, and excipient granules subsequently added to the aqueous solution. As another example, each of the excipient granules, the immediate release pharmaceutical granules, and the modified release pharmaceutical granules can be added in any order, simultaneously, or independently to an aqueous solution such as water to provide an oral pharmaceutical composition.

An oral pharmaceutical composition can comprise a viscous solution and a crystalline 4-((L-valyl)oxy)butanoic acid salt dissolved in the viscous solution and/or pharmaceutical granules comprising a crystalline 4-((L-valyl)oxy)butanoic acid salt suspended in the viscous solution.

An oral pharmaceutical composition can comprise an immediate release component and a modified release component. An immediate release component can comprise a crystalline 4-((L-valyl)oxy)butanoic acid salt dissolved in the viscous aqueous solution, immediate release pharmaceutical granules suspended in the viscous solution, or a combination thereof. A modified release component can comprise modified release pharmaceutical granules suspended in the viscous solution.

An immediate release component and a modified release component can comprise a crystalline 4-((L-valyl)oxy)butanoic acid salt.

An oral pharmaceutical composition can have a viscosity, for example, from 50 cP to 1,100 cP or from 50 cP to 600 cP at a temperature from 20° C. to 25° C., such as 23° C., determined using a No. 62 spindle at a speed of 12 rpm according to USP <912>, Method 1c. An oral pharmaceutical composition can have a viscosity, for example, greater than 50 cP, greater than 100 cP, greater than 200 cP, greater than 400 cP, greater than 800 cP or greater than 800 cP at a temperature from 20° C. to 25° C., such as 23° C., determined using a No. 62 spindle at a speed of 12 rpm according to USP <912>, Method 1C. An oral pharmaceutical composition can have a viscosity, for example, of less than 1,000 cP, less than 800 cP, less than 600 cP, less than 400 cP, less than 200 cP, or less than 100 cP, at a temperature from 20° C. to 25° C., such as 23° C., determined using a No. 62 spindle at a speed of 12 rpm according to USP <912>, Method 1C.

An oral pharmaceutical composition provided by the present disclosure can have a viscosity, for example, from 350 cp to 550 cp in 30 seconds after mixing with water, from 775 cP to 975 cp in 60 seconds, and from 950 cP to 1,150 cP after mixing with water, where the viscosity is determined according to USP <912> Method 1C, using a No. 62 spindle at a speed of 12 rpm, at a temperature of 23° C., and a concentration of 40 g/L.

An oral pharmaceutical composition provided by the present disclosure can have a viscosity, for example, from 400 cp to 500 cp in 30 seconds after mixing with water, from 825 cP to 925 cp in 60 seconds, and from 1,000 cP to 1,100 cP after mixing with water, where the viscosity is determined according to USP <912> Method 1C, using a No. 62 spindle at a speed of 12 rpm, at a temperature of 23° C., and a concentration of 40 g/L.

An oral pharmaceutical composition can have a viscosity, for example, from 10 cP to 1,000 cP or from 20 cP to 800 cP at a temperature from 20° C. to 25° C. such as 23° C. An oral pharmaceutical composition can have a viscosity, for example, greater than 10 cP, greater than 20 cP, greater than 50 cP, greater than 100 cP, greater than 200 cP, greater than 400 cP, greater than 600 cP, or greater than 800 cP at a temperature from 20° C. to 25° C. such as 23° C. An oral pharmaceutical composition can have a viscosity, for example, of less than 1,000 cP, less than 800 cP, less than 600 cP, less than 400 cP, or less than 200 cP, at a temperature from 20° C. to 25° C. such as 23° C. Viscosity is determined using a Brookfield Digital Viscometer Model LVDV-I+ equipped with a Brookfield SC4-18 spindle at a spindle speed of 30 rpm. The oral pharmaceutical composition can be prepared by combining and mixing, for example, from 10 g to 20 g of a pharmaceutical composition with from 30 mL to 50 mL of water. The oral pharmaceutical composition can be prepared by combining and mixing, for example, from 12 g to 18 g of a pharmaceutical composition with from 35 mL to 45 mL of water. The oral composition can have a concentration of the pharmaceutical composition, for example, from 0.20 g/mL to 0.70 g/mL, such as from 0.30 g/mL to 0.42 g/mL, or from 0.32 g/mL to 0.40 g/mL.

Following reconstitution in water, an oral pharmaceutical composition can be pourable and have a viscosity similar to that of drinkable yogurt.

A pharmaceutical composition can comprise excipient granules and a pharmaceutical granulation comprising an immediate-release pharmaceutical granules and modified-release pharmaceutical granules. When combined with a suitable amount of water, the excipient granules can disintegrate to cause the viscosity of the solution to increase, the immediate release pharmaceutical granules can dissolve to provide the active pharmaceutical ingredient dissolved in the viscous solution, and the modified-release pharmaceutical granules can be suspended and dispersed in the viscous solution.

The modified-release pharmaceutical granules can be configured to retain the physical integrity of the modified release granules and dissolve and/or release the crystalline 4-((L-valyl)oxy)butanoic acid salt over time in the gastrointestinal tract with a release profile determined by the constituents and the coating of the modified-release pharmaceutical granules or combination of modified-release pharmaceutical granules.

A pharmaceutical composition comprising about 7.6 wt % of excipient granules, about 25.2 wt % of immediate release pharmaceutical granules, and about 67.2 wt % of modified release pharmaceutical granules can exhibit a crystalline 4-((L-valyl)oxy)butanoic acid salt dissolution profile wherein from 26% to 46% is released within 0.5 hours, from 41% to 61% is released within 1 hour, from 63% to 83% is released within 2 hours, and from 73% to 93% is released within 4 hours.

Pharmaceutical compositions provided by the present disclosure can be provided, for example, as sachets containing pharmaceutical granules comprising crystalline 4-((L-valyl)oxy)butanoic acid, such as a combination of immediate release granules and modified release granules. A sachet can be provided in different doses of a crystalline 4-((L-valyl)oxy)butanoic acid salt such as from 0.5 g to 30 g, or from 1 g to 20 g of a crystalline 4-((L-valyl)oxy)butanoic acid salt. The pharmaceutical granules can be combined, for example, with water to provide an orally ingestible dosage form.

A pharmaceutical composition provided by the present disclosure can be included in a kit that can be used to administer a crystalline 4-((L-valyl)oxy)butanoic acid salt to a patient for therapeutic purposes. A kit can include a pharmaceutical composition, instructions for preparing an oral pharmaceutical composition suitable for oral administration to a patient, and instructions for administering the oral pharmaceutical composition to the patient. The kit can comprise a pharmaceutical composition provided by the present disclosure and instructions for combining the contents of the kit with an aqueous solution to provide an oral pharmaceutical composition and with instructions or administering the oral pharmaceutical composition to a patient.

A kit can be used, for example, to treat a sleep disorder. A kit can comprise a crystalline 4-((L-valyl)oxy)butanoic acid hydrochloride, a pharmaceutically acceptable vehicle for administering crystalline 4-((L-valyl)oxy)butanoic acid salt, and instructions for administering a crystalline 4-((L-valyl)oxy)butanoic acid salt to a patient

A kit can comprise a primary package and a secondary package containing a pharmaceutical composition.

Pharmaceutical compositions or granules can be included in primary package such as a container, sachet, blister pack, package, or dispenser together with instructions for preparation of the oral pharmaceutical composition and administration.

Instructions supplied with a kit may be printed and/or supplied, for example, as an electronic-readable medium, a video cassette, an audiotape, a flash memory device, or may be published on an internet web site or distributed to a patient and/or health care provider as an electronic communication.

A pharmaceutical product can comprise a compartment containing the excipient granules and a second compartment containing the pharmaceutical granules where the compartments are configured to be mixed before or during combination with an aqueous solution.

A pharmaceutical product provided by the present disclosure can comprise a first package comprising excipient granules provided by the present disclosure and a second package comprising pharmaceutical granules.

A pharmaceutical product can comprise a sachet that contains both excipient granules provided by the present disclosure and pharmaceutical granules.

A pharmaceutical product can comprise, for example, from 0.5 g to 30 g of a combination of excipient granules and pharmaceutical granules, such as from 1 g to 25 g, from 1 g to 20 g, from 2 g to 15 g, from 2 g to 10 g, from 2 g to 5 g, or from 1 g to 5 g of a combination of excipient granules and pharmaceutical granules.

A pharmaceutical product provided by the present disclosure can comprise a unit dose of a crystalline 4-((L-valyl)oxy)butanoic acid salt.

A pharmaceutical composition or oral dosage form comprising a crystalline 4-((L-valyl)oxy)butanoic acid salt can comprise an immediate release component and a modified release component.

For example, an immediate release component can comprise a solution comprising a 4-((L-valyl)oxy)butanoic acid salt or immediate release granules comprising a crystalline 4-((L-valyl)oxy)butanoic acid salt.

For example, a modified release component can comprise modified release granules comprising a crystalline 4-((L-valyl)oxy)butanoic acid salt

A combined release oral dosage form can comprise modified release granules comprising a crystalline 4-((L-valyl)oxy)butanoic acid salt suspended in a solution comprising dissolved 4-((L-valyl)oxy)butanoic acid hydrochloride and/or partially dissolved immediate release granules comprising a crystalline 4-((L-valyl)oxy)butanoic acid salt.

An oral dosage form can comprise granules. For example, an oral dosage form can comprise immediate release granules and modified release granules. For example, an oral dosage form can comprise a suspension containing a crystalline 4-((L-valyl)oxy)butanoic acid salt. An oral dosage form can comprise a suspension of immediate release granules comprising a crystalline 4-((L-valyl)oxy)butanoic acid salt and modified release granules comprising a crystalline 4-((L-valyl)oxy)butanoic acid salt. An oral dosage form can comprise a crystalline 4-((L-valyl)oxy)butanoic acid salt dissolved in a solution such as an aqueous solution and modified release granules comprising a crystalline 4-((L-valyl)oxy)butanoic acid salt suspended in the solution.

A pharmaceutical composition and oral dosage form provided by the present disclosure can comprise any suitable excipient, salt, acid, pH-mediating, adjusting or buffering compound or agent, flavoring, solution, solvent, dispersion, glycerol, glycol, oil, antibacterial and antifungal agent, antibiotic and antihistamine, binder, disintegrating agent, lubricant, sweetening agent, or any other suitable additive or ingredient.

Examples of suitable oral formulations and oral dosage forms are disclosed in U.S. Application Publication No. 2021/0393537, U.S. Pat. No. 11,304,906, U.S. Application Publication No. 2022/0023247, U.S. Pat. Nos. 11,395,801, and 11,510,892, each of which is incorporated by reference in its entirety.

A unit dose of a crystalline 4-((L-valyl)oxy)butanoic acid salt can be provided in one or more packages such as one or more sachets.

A pharmaceutical product provided by the present disclosure can comprise more than one unit dose of a crystalline 4-((L-valyl)oxy)butanoic acid salt. Each of the one or more unit doses can be provided in separate packages such as in separate sachets.

The unit dose can be suitable for treating a disease of a patient.

An oral pharmaceutical composition provided by the present disclosure can provide a therapeutically effective amount of γ-hydroxybutyric acid over a period of time in the plasma of a patient. For example, an oral formulation provided by the present disclosure can provide a therapeutically effective amount of γ-hydroxybutyric acid over a period of 3 hours, 6 hours, 8 hours, or 10 hours.

An oral pharmaceutical composition provided by the present disclosure can provide a therapeutically effective amount of γ-hydroxybutyric acid over a period from 4 hours to 12 hours, from 4 hours to 10 hours, or from 4 hours to 8 hours.

An oral pharmaceutical composition provided by the present disclosure can provide a therapeutically effective amount of γ-hydroxybutyric acid over a duration, for example, from 1 hour to 12 hours following oral administration, from 2 hours to 10 hours or from 4 hours to 8 hours following oral administration.

An oral pharmaceutical composition provided by the present disclosure can be a once nightly formulation. For a once nightly formulation, a patient can administer a dose of a crystalline 4-((L-valyl)oxy)butanoic acid salt and/or γ-hydroxybutyric acid as a prodrug provided by the present disclosure before going to bed and sleep through the night such as for 6 hours or for 8 hours without having to administer a second dose during the night.

An oral pharmaceutical composition provided by the present disclosure can provide a therapeutically effective amount of γ-hydroxybutyric acid in the plasma of a patient. An oral pharmaceutical composition provided by the present disclosure can provide a therapeutically effective amount of γ-hydroxybutyric acid in the plasma of a patient for a period of 4 hours, 6 hours, 8 hours, or 10 hours following oral administration of the modified release oral pharmaceutical composition.

An oral pharmaceutical composition provided by the present disclosure can provide a plasma concentration of γ-hydroxybutyric acid, for example, greater than 10 μg/mL for more than 4 hours, for more than 6 hours, for more than 8 hours, or for more than 10 hours following oral administration of the oral pharmaceutical composition.

An oral pharmaceutical composition provided by the present disclosure can provide a plasma concentration of γ-hydroxybutyric acid, for example, greater than 15 μg/mL for more than 4 hours, for more than 6 hours, for more than 8 hours, or for more than 10 hours following oral administration of the oral pharmaceutical composition.

An oral formulation provided by the present disclosure can provide a therapeutically effective amount of Cₘₐₓ to Cₘᵢₙ ratio of γ-hydroxybutyric acid in the plasma of a patient, for example, from less than 3 or less than 2 for a duration of 4 hours, 6 hours, 8 hours, or 10 hours following oral administration of the modified release oral pharmaceutical composition.

An oral pharmaceutical composition provided by the present disclosure can comprise a crystalline 4-((L-valyl)oxy)butanoic acid salt and can comprise, for example, 0.5 g-equivalents of γ-hydroxybutyric acid, 1 g-equivalents, 2 g-equivalents, 3 g-equivalents, 4 g-equivalents, 5 g-equivalents, 6 g-equivalents, 7 g-equivalents, 8 g-equivalents, 9 g-equivalents, 10 g-equivalents, 11 g-equivalents, or 12 g-equivalents of γ-hydroxybutyric acid. An oral pharmaceutical composition provided by the present disclosure can comprise a crystalline 4-((L-valyl)oxy)butanoic acid salt and can comprise, for example, from 0.5 g-equivalents to 12 g-equivalents of γ-hydroxybutyric acid, from 1 g-equivalents to 12 g-equivalents, from 2 g-equivalents to 12 g-equivalents, from 3 g-equivalents to 11 g-equivalents, from 4 g-equivalents to 10 g-equivalents, or from 5 g-equivalents to 9 g-equivalents of γ-hydroxybutyric acid. An oral pharmaceutical composition provided by the present disclosure can comprise, for example, greater than 0.5 g-equivalents of γ-hydroxybutyric acid, greater than 1 g-equivalents, greater than 3 g-equivalents, greater than 5 g-equivalents, greater than 7 g-equivalents, greater than 9 g-equivalents, or greater than 11 g-equivalents of γ-hydroxybutyric acid.

An oral pharmaceutical composition provided by the present disclosure can comprise a suspension such as an aqueous suspension of immediate release pharmaceutical granules and modified release granules comprising a crystalline 4-((L-valyl)oxy)butanoic acid salt. Immediate release pharmaceutical granules and the core of the modified release pharmaceutical granules can comprise greater than 90 wt % such as greater than 94 wt %, or greater than 96 wt % of a crystalline 4-((L-valyl)oxy)butanoic acid salt, where wt % is based on the total weight of the immediate release pharmaceutical granules or the core of the modified release pharmaceutical granules.

An oral pharmaceutical composition provided by the present disclosure can be provided, for example, as a sachet or package containing an immediate release component such as an immediate release pharmaceutical granules comprising a crystalline 4-((L-valyl)oxy)butanoic acid salt and a modified release component such as modified release pharmaceutical granules comprising a crystalline 4-((L-valyl)oxy)butanoic acid salt. A sachet or package can be provided in different dose equivalents of γ-hydroxybutyric acid such as from 0.5 g-equivalents to 20 g-equivalents of γ-hydroxybutyric acid.

An oral dosage form such as coated granules comprising immediate release granules and modified release granules can be combined, for example, with water to provide an orally ingestible dosage form.

Following oral administration of an oral formulation provided by the present disclosure and absorption of a crystalline 4-((L-valyl)oxy)butanoic acid salt in the gastrointestinal tract can be metabolized in the systemic circulation of a patient to provide γ-hydroxybutyric acid.

Methods provided by the present disclosure include providing a therapeutically effective amount of γ-hydroxybutyric acid in the systemic circulation of a patient for treating a disease or disorder in a patient or a symptom of a disease or disorder in a patient comprising administering to a patient an oral formulation provided by the present disclosure comprising a crystalline 4-((L-valyl)oxy)butanoic acid salt.

A suitable dose of a crystalline 4-((L-valyl)oxy)butanoic acid salt can provide, for example, a dose of from 1 gram-equivalents to 12 gram-equivalents of γ-hydroxybutyric acid, such as a dose from 1 gram-equivalents to 10 gram-equivalents, from 2 gram-equivalents to 9 gram-equivalents, from 3 gram-equivalents to 8 gram-equivalents, or from 4 gram-equivalents to 7 gram-equivalents of γ-hydroxybutyric acid.

It will be understood, however, that the specific dose level and frequency of dosage for any particular patient may be varied and will depend upon a variety of factors including: the metabolic stability and length of action, the age, body weight, general health, gender, diet, mode and time of administration, rate of excretion, drug combination, the severity of the particular condition, and the host undergoing therapy.

When initiating treatment with a crystalline 4-((L-valyl)oxy)butanoic acid salt, titration up to an adequate plasma concentration can facilitate obtaining positive results and avoid adverse effects. For example, a dose can be 4.5 g-equivalents of γ-hydroxybutyric acid divided into 2 equal doses of 2.25 g, the first taken at bedtime and the second taken 2.5 hours to 4 hours later. The starting dose can be decreased to 3.0 g/day or increased to as high as 9.0 g/day in increments of 1.5 g/day (0.75 g per dose). A dose can provide an effective eight hours of sleep but, at the end of eight hours, little of the γ-hydroxybutyric acid will remain in the plasma of a patient.

An oral pharmaceutical formulation provided by the present disclosure can be configured to provide for once a night dosing, once a day dosing (QD), twice a day dosing (BID), three times a day dosing (TID), or four times a day dosing (QID). For example, a pharmaceutical composition comprising modified release granules can release substantially 100% of a crystalline 4-((L-valyl)oxy)butanoic acid salt over a 24-hour duration, a 12-hour duration, an 8-hour duration, or a 4-hour duration.

A crystalline 4-((L-valyl)oxy)butanoic acid salt is a prodrug of γ-hydroxybutyric acid. A crystalline 4-((L-valyl)oxy)butanoic acid salt can be used to treat any disease or disorder that is known to be treated by γ-hydroxybutyric acid or is determined to be treated by γ-hydroxybutyric acid.

For example, crystalline 4-((L-valyl)oxy)butanoic acid hydrochloride can be used to treat narcolepsy, excessive daytime sleepiness, cataplexy, excessive daytime sleepiness associated with narcolepsy, excessive daytime sleepiness associated with Parkinson's disease, excessive daytime sleepiness associated with multiple sclerosis, cataplexy associated with narcolepsy, fatigue, fatigue associated with Parkinson's disease, fatigue associated with multiple sclerosis, and fibromyalgia.

A crystalline 4-((L-valyl)oxy)butanoic acid salt can be used to treat REM sleep behavior disorder, spasmodic dystonia, schizophrenia, insomnia, insomnia associated with schizophrenia, idiopathic hypersomnia, chronic fatigue syndrome, cluster headache, symptoms of Alzheimer's disease, essential tremor, post-traumatic stress syndrome, insomnia associated with post-traumatic stress syndrome, and anxiety.

A crystalline 4-((L-valyl)oxy)butanoic acid salt can be used to treat excessive daytime sleepiness associated with narcolepsy, excessive daytime sleepiness associated with Parkinson's disease, excessive daytime sleepiness associated with multiple sclerosis, cataplexy associated with narcolepsy, fatigue in a patient with Parkinson's disease, fatigue in a patient with multiple sclerosis, or fibromyalgia.

A crystalline 4-((L-valyl)oxy)butanoic acid salt and pharmaceutical compositions thereof can be used to treat idiopathic hypersomnia.

A crystalline 4-((L-valyl)oxy)butanoic acid salt and pharmaceutical compositions thereof can be used to treat as sleep disorder associated with Parkinson's disease.

A crystalline 4-((L-valyl)oxy)butanoic acid salt and pharmaceutical compositions thereof can be used to treat disturbed nocturnal sleep.

A crystalline 4-((L-valyl)oxy)butanoic acid salt and pharmaceutical compositions thereof can be used to treat a sleep disorder such as apnea, a sleep time disturbance, narcolepsy, cataplexy, sleep paralysis, hypnagogic hallucination, sleep arousal, insomnia, and nocturnal myoclonus.

A crystalline 4-((L-valyl)oxy)butanoic acid salt or a pharmaceutical composition thereof can be used to treat a disease selected from narcolepsy, cataplexy, cataplexy with narcolepsy, excessive daytime sleepiness, a sleep disorder associated with Parkinson's disease, symptoms of Parkinson's disease, a neurodegenerative disease, sleep disturbance syndrome, fatigue, improving nocturnal sleep, hypnagogic hallucinations, sleep paralysis, fragmented sleep, alcohol withdrawal and dependence, obstructive sleep apnea syndrome, insomnia, insomnia associated with schizophrenia, sleep ignition and maintenance disorders, chronic fatigue syndrome, essential tremor, hemiplegia in patients with alternating hemiplegia of childhood, sedative abuse, and binge eating disorder.

For certain methods of treatment the effectiveness of the treatment can be measured by one or more of the following criteria: increase in the mean sleep latency such as determined the Maintenance of Wakefulness Test (MWT); improvement in the Clinical Global Impression (CGI) rating of sleepiness; decrease in the number of cataplexy attacks (NCA) such as determined from the cataplexy frequency item in the Sleep and Symptoms Daily Diary; decrease in disturbed nocturnal sleep (DNS), the disturbed nocturnal events or the adverse respiratory events such as determined by polysomnographic (PSG) measures of sleep fragmentation; decrease in excessive daytime sleepiness (EDS) such as measured by patient report via the Epworth Sleepiness Scale (ESS); decrease in daytime sleepiness as measured by the Maintenance of Wakefulness Test based on EEG measures of wakefulness; decrease PSG transitions from N/2 to N/3 and REM sleep to wake and N1 sleep such a determined as described in the AASM Manual for the Scoring of Sleep and Associated Events; decrease in the number of arousals or awakenings such as determined from a PSG as defined by the American Academy of Sleep Medicine; improvement in sleep quality such as determined using (i) the Sleep and Symptom Daily Diary, (ii) Visual Analog Scale (VAS) for sleep quality and sleep diary, and/or (iii) VAS for the refreshing nature of sleep; and decrease in the Hypnagogic Hallucinations (HH) or sleep paralysis (SP) symptoms in NT1 narcolepsy patients such as measured by the Sleep and Symptom Daily Diary.

Type 1 Narcolepsy (NT1) refers to narcolepsy characterized by excessive daytime sleepiness (“EDS”) and cataplexy. Type 2 Narcolepsy (NT2) refers to narcolepsy characterized by excessive daytime sleepiness without cataplexy. A diagnosis of narcolepsy (with or without cataplexy) can be confirmed by one or a combination of (i) an overnight polysomnogram (PSG) and a Multiple Sleep Latency Test (MSLT) performed within the last 2 years, (ii) a full documentary evidence confirming diagnosis from the PSG and MSLT from a sleep laboratory must be made available, (iii) current symptoms of narcolepsy including: current complaint of EDS for the last 3 months (Epworth Sleepiness Scale (ESS) greater than 10), (iv) mean Maintenance of Wakefulness Test (MWT) less than 8 minutes, (v) mean number of cataplexy events of 8 per week on baseline Sleep/Cataplexy Diary, and/or (vi) presence of cataplexy for the last 3 months and 28 events per week during screening period.

### Aspects of the Invention

The invention is further defined by the following aspects.

Aspect 1. A compound selected from crystalline 4-((L-valyl)oxy)butanoic acid hydrochloride and crystalline 4-((L-valyl)oxy)butanoic acid oxalate.

Aspect 2. The compound of aspect 1, wherein the compound is crystalline 4-((L-valyl)oxy)butanoic acid hydrochloride having the structure of Formula (1):

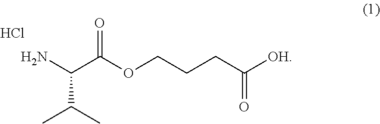

Aspect 3. The compound of aspect 2, wherein the compound is characterized by an XRPD pattern comprising characteristic diffraction peaks at least at 8.05°±0.20°, 21.66°±0.20°, and 24.09°±0.20° expressed as 2θ/θ angles determined using Cu-Kα radiation.

Aspect 4. The compound of aspect 2, wherein the compound is characterized by an XRPD pattern comprising characteristic diffraction peaks at least at 8.05°±0.10°, 21.66°±0.10°, and 24.09°±0.100 expressed as 2θ/θ angles determined using Cu-Kα radiation.

Aspect 5. The compound of aspect 2, wherein the compound is characterized by an XRPD pattern comprising characteristic diffraction peaks at least at 8.05°±0.20°, 10.72°±0.20°, 16.12°±0.20°, 21.66°±0.20°, 24.09°±0.20°, and 27.38°±0.20° expressed as 2θ/θ angles determined using Cu-Kα radiation.

Aspect 6. The compound of aspect 2, wherein the compound is characterized by an XRPD pattern comprising characteristic diffraction peaks at least at 8.05°±0.10°, 10.72°±0.10°, 16.12°±0.10°, 21.66°±0.10°, 24.09°±0.10°, and 27.38°±0.10° expressed as 2θ/θ angles determined using Cu-Kα radiation.

Aspect 7. The compound of aspect 2, wherein the compound is characterized by an XRPD pattern comprising characteristic diffraction peaks at least at 8.05°±0.20°, 10.72±0.20°, 16.12°±0.20°, 16.53±0.20°, 17.62°±0.20°, 21.66°±0.20°, 24.09°±0.20°, 26.03°±0.20°, 27.38°±0.20°, and 30.09°±0.20° expressed as 2θ/θ angles determined using Cu-Kα radiation.

Aspect 8. The compound of aspect 2, wherein the compound is characterized by an XRPD pattern comprising characteristic diffraction peaks at least at 8.05°±0.10°, 10.72±0.10°, 16.12°±0.10°, 16.53±0.10°, 17.62°±0.10°, 21.66°±0.10°, 24.09°±0.10°, 26.03°±0.10°, 27.38°±0.10°, and 30.09°±0.100 expressed as 2θ/θ angles determined using Cu-Kα radiation.

Aspect 9. The compound of aspect 2, wherein the compound is characterized by an XRPD pattern substantially as shown in FIG. 1.

Aspect 10. The compound of any one of aspects 2 to 9, wherein the compound has a DSC profile characterized by an exotherm with an onset temperature at 97.04° C.±2° C., and a peak at 99.19° C.±2° C.

Aspect 11. The compound of any one of aspects 2 to 10, wherein the compound is characterized by DSC profile substantially as shown in FIG. 2.

Aspect 12. The compound of any one of aspects 2 to 11, wherein the compound is stable in an aqueous solution at a pH from 1.2 to 8.0 at a temperature of 37° C., for up to 240 hours or in dimethyl sulfoxide (DMSO) for up to 96 hours.

Aspect 13. The compound of any one of aspects 2 to 12, wherein the compound absorbs less than 2 wt % water at conditions of 25° C./60% RH for 36 months.

Aspect 14. The compound of any one of aspects 2 to 13, wherein the compound has an impurity content of less than 1 wt % at conditions of 25° C./60% RH for 36 months.

Aspect 15. The compound of any one of aspects 2 to 14, wherein the compound has a water content of less than 5 wt % and an impurity content of less than 5 wt % after storage at 25° C./60% RH for 6 months, wherein wt % is based on the total weight of the compound, water, and impurity; the water content is determined using Karl Fischer analysis, and the impurity content is determined using high pressure liquid chromatography.

Aspect 16. The compound of aspect 1, wherein the compound is crystalline 4-((L-valyl)oxy)butanoic acid oxalate having the structure of Formula (2):

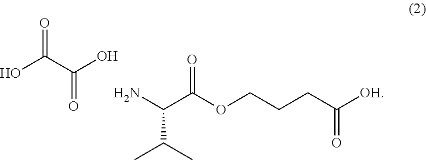

Aspect 17. The compound of aspect 16, wherein the compound is characterized by an XRPD pattern comprising characteristic diffraction peaks at least at 6.48°±0.20°, 9.44°±0.20°, 18.20°±0.20°, and 27.24°±0.20°, expressed as 2θ/θ angles determined using Cu-Kα radiation.

Aspect 18. The compound of aspect 16, wherein the compound is characterized by an XRPD pattern comprising characteristic diffraction peaks at least at 6.48°±0.10°, 9.44°±0.10°, 18.20°±0.10°, and 27.24°±0.10°, expressed as 2θ/θ angles determined using Cu-Kα radiation.

Aspect 19. The compound of aspect 16, wherein the compound is characterized by an XRPD pattern comprising characteristic diffraction peaks at least at 6.48°±0.20°, 9.44°±0.20°, 11.04°±0.20°, 17.03°±0.20°, 18.20°±0.20°, 27.24°±0.20°, and 28.86°±0.20°, expressed as 2θ/θ angles determined using Cu-Kα radiation.

Aspect 20. The compound of aspect 16, wherein the compound is characterized by an XRPD pattern comprising characteristic diffraction peaks at least at 6.48°±0.20°, 9.44°±0.10°, 11.04°±0.10°, 17.03°±0.10°, 18.20°±0.10°, 27.24°±0.10°, and 28.86°±0.10°, expressed as 2θ/θ angles determined using Cu-Kα radiation.

Aspect 21. The compound of aspect 16, wherein the compound is characterized by an XRPD pattern comprising characteristic diffraction peaks at least at 6.48°±0.20°, 9.44°±0.20°, 11.04°±0.20°, 17.03°±0.20°, 18.20°±0.20°, 18.73°±0.20°, 19.08°±0.20°, 21.73°±0.20°, 22.46°±0.20°, 27.24°±0.20°, 28.86°±0.20°, and 29.45°±0.20°, expressed as 2θ/θ angles determined using Cu-Kα radiation.

Aspect 22. The compound of aspect 16, wherein the compound is characterized by an XRPD pattern comprising characteristic diffraction peaks at least at 6.48°±0.10°, 9.44°±0.10°, 11.04°±0.10°, 17.03°±0.10°, 18.20°±0.10°, 18.73°±0.10°, 19.08°±0.10°, 21.73°±0.10°, 22.46°±0.10°, 27.24°±0.10°, 28.86°±0.10°, and 29.45°±0.10°, expressed as 2θ/θ angles determined using Cu-Kα radiation.

Aspect 23. The compound of aspect 16, wherein the compound is characterized by an XRPD pattern substantially as shown in FIG. 3.

Aspect 24. The compound of any one of aspects 16 to 23, wherein the compound is stable in an aqueous solution at a pH from 1.2 to 8.0 at a temperature of 37° C., for up to 240 hours or in dimethyl sulfoxide (DMSO) for up to 96 hours.

Aspect 25. The compound of any one of aspects 16 to 24, wherein the compound absorbs less than 2 wt % water at conditions of 25° C./60% RH for 36 months.

Aspect 26. The compound of any one of aspects 16 to 25, wherein the compound has an impurity content of less than 1 wt % at conditions of 25° C./60% RH for 36 months.

Aspect 27. The compound of any one of aspects 16 to 26, wherein the compound has a water content of less than 5 wt % and an impurity content of less than 5 wt % after storage at 25° C./60% RH for 6 months, wherein wt % is based on the total weight of the compound, water, and impurity; the water content is determined using Karl Fischer analysis, and the impurity content is determined using high pressure liquid chromatography.

Aspect 28. Granules comprising the compound of any one of aspects 1 to 27.

Aspect 29. The granules of aspect 28, wherein the granules have an average particle size from 100 μm to 600 μm.

Aspect 30. The granules of any one of aspects 28 to 29, wherein the granules comprise greater than 60 wt % of the compound, wherein wt % is based on the total weight of the granules.

Aspect 31. The granules of any one of aspects 28 to 30, wherein the granules comprise immediate release granules.

Aspect 32. The granules of aspect 31, wherein the immediate release granules have a homogeneous composition.

Aspect 33. The granules of any one of aspects 31 to 32, wherein the immediate release granules comprise a water-soluble polymer and an antistatic agent.

Aspect 34. The granules of any one of aspects 31 to 33, wherein the immediate release granules comprise greater than 70 wt % of the compound; and wt % is based on the total weight of the immediate release granules.

Aspect 35. The granules of any one of aspects 28 to 34, wherein the granules comprise a plurality of modified release granules.

Aspect 36. The granules of aspect 35, wherein the modified release granules comprise greater than 60 wt % of the compound and wt % is based on the total weight of the modified release granules.

Aspect 37. The granules of any one of aspects 35 to 36, wherein the modified release granules comprise immediate release granules coated with a modified release coating.

Aspect 38. The granules of any one of aspects 35 to 37, wherein the modified release coating comprises a water-soluble polymer, a water-insoluble polymer, an antistatic agent, and a plasticizer.

Aspect 39. The granules of any one of aspects 28 to 30, wherein the granules comprise the granules of any one of aspects 31 to 38.

Aspect 40. A pharmaceutical composition comprising the compound of any one of aspects 1 to 27 or the granules of any one of aspects 28 to 39.

Aspect 41. The pharmaceutical composition of aspect 40, wherein the pharmaceutical composition comprises a therapeutically effective amount of the compound for treating a disease in a patient, wherein the disease is selected from narcolepsy, cataplexy, cataplexy with narcolepsy, excessive daytime sleepiness, a sleep disorder associated with Parkinson's disease, Parkinson's disease, a neurodegenerative disease, idiopathic hypersomnia, sleep disturbance syndrome, fatigue, improving nocturnal sleep, hypnagogic hallucinations, sleep paralysis, fragmented sleep, alcohol withdrawal and dependence, obstructive sleep apnea syndrome, insomnia, insomnia associated with schizophrenia, sleep ignition and maintenance disorders, chronic fatigue syndrome.

Aspect 42. The pharmaceutical composition of any one of aspects 40 to 41, wherein the pharmaceutical composition comprises an oral formulation.

Aspect 43. The pharmaceutical composition of any one of aspects 40 to 42, wherein the pharmaceutical composition comprises from 1 gram-equivalents to 10 gram-equivalents of γ-hydroxybutyric acid.

Aspect 44. The pharmaceutical composition of any one of aspects 40 to 43, wherein the pharmaceutical composition comprises from 1 gram to 20 grams of the compound.

Aspect 45. The pharmaceutical composition of any one of aspects 40 to 44, wherein the pharmaceutical composition comprises an immediate release component.

Aspect 46. The pharmaceutical composition of aspect 45, wherein the immediate release component comprises the granules of any one of aspects 31 to 34.

Aspect 47. The pharmaceutical composition of any one of aspects 45 and 46, wherein the immediate release component comprises a solution comprising the compound.

Aspect 48. The pharmaceutical composition of any one of aspects 40 to 47, wherein the pharmaceutical composition comprises a modified release component.

Aspect 49. The pharmaceutical composition of aspect 48, wherein the modified release component comprises the granules of any one of aspects 35 to 38.

Aspect 50. The pharmaceutical composition of any one of aspects 48 to 49, wherein the modified release component comprises the granules of any one of aspects 35 to 38 suspended in a viscous aqueous solution.

Aspect 51. The pharmaceutical composition of any one of aspects 30 to 44, wherein the pharmaceutical composition comprises the immediate release component of any one of aspects 40 to 42 and the modified release component of any one of aspects 48 to 50.

Aspect 52. An oral dosage form comprising the compound of any one of aspects 1 to 27, the granules of any one of aspects 28 to 40, or the pharmaceutical composition of any one of aspects 33 to 44.

Aspect 53. The oral dosage form of aspect 52, wherein the oral dosage form comprises from 1 gram-equivalents to 10 gram-equivalents of γ-hydroxybutyric acid.

Aspect 54. The oral dosage form of any one of aspects 52 to 53, wherein the oral dosage form comprises from 1 gram to 20 grams of the compound.

Aspect 55. The oral dosage form of any one of aspects 52 to 54, wherein the oral dosage form comprises an immediate release component and a modified release component.

Aspect 56. The oral dosage form of any one of aspects 52 to 55, wherein the pharmaceutical composition comprises an immediate release component.

Aspect 57. The oral dosage form of aspect 56, wherein the immediate release component comprises a solution comprising the compound.

Aspect 58. The oral dosage form of aspect 56, wherein the immediate release component comprises immediate release granules comprising the compound.

Aspect 59. The oral dosage form of any one of aspects 52 to 58, wherein the pharmaceutical composition comprises a modified release component.

Aspect 60. The oral dosage form of aspect 59, wherein the modified release component comprises modified release granules comprising the compound de.

Aspect 61. The oral dosage form of aspect 59, wherein the oral dosage form comprises modified release granules comprising the compound suspended in a solution comprising the compound.

Aspect 62. A kit comprising the compound of any one of aspects 1 to 27, the granules of any one of aspects 28 to 39, or the pharmaceutical composition of any one of aspects 40 to 51

Aspect 63. The kit of aspect 62, wherein the kit comprises: an immediate release component comprising the compound; and a modified release component comprising the compound.

Aspect 64. The kit of 63, wherein the immediate release component comprises immediate release granules comprising the compound; and the modified release component comprises modified release granules comprising the compound.

Aspect 65. The kit of any one of aspects 62 to 64, wherein the kit comprises a sachet containing the compound, the granules, or the pharmaceutical composition.

Aspect 66. A method of treating a disease in a patient comprising administering to a patient in need of such treatment a therapeutically effective amount of the compound of any one of aspects 1 to 27, the granules of any one of aspects 28 to 39, the pharmaceutical composition of any one of aspects 40 to 51, or the oral dosage form of any one of aspects 52 to 61, wherein the disease is capable of being treated with γ-hydroxybutyric acid.

Aspect 67. A method of treating a disease in a patient comprising administering to a patient in need of such treatment a therapeutically effective amount of the compound of any one of aspects 1 to 27, the granules of any one of aspects 28 to 39, the pharmaceutical composition of any one of aspects 40 to 51, or the oral dosage form of any one of aspects 52 to 61, wherein the disease is selected from narcolepsy, cataplexy, cataplexy with narcolepsy, excessive daytime sleepiness, a sleep disorder associated with Parkinson's disease, Parkinson's disease, a neurodegenerative disease, sleep disturbance syndrome, fatigue, improving nocturnal sleep, hypnagogic hallucinations, sleep paralysis, fragmented sleep, alcohol withdrawal and dependence, obstructive sleep apnea syndrome, insomnia, insomnia associated with schizophrenia, sleep ignition and maintenance disorders, chronic fatigue syndrome, essential tremor, hemiplegia in patients with alternating hemiplegia of childhood, sedative abuse, and binge eating disorder.

Aspect 68. A method of treating fatigue or excessive daytime sleepiness associated with narcolepsy comprising orally administering to a patient in need of such treatment a therapeutically effective amount of the compound of any one of aspects 1 to 27, the granules of any one of aspects 28 to 39, the pharmaceutical composition of any one of aspects 40 to 51, or the oral dosage form of any one of aspects 52 to 61.

Aspect 69. A method of treating narcolepsy, excessive daytime sleepiness, cataplexy, excessive daytime sleepiness associated with narcolepsy, excessive daytime sleepiness associated with Parkinson's disease, excessive daytime sleepiness associated with multiple sclerosis, cataplexy associated with narcolepsy, fatigue, fatigue associated with Parkinson's diseases, fatigue associated with multiple sclerosis, idiopathic hypersomnia, or fibromyalgia comprising orally administering to a patient in need of such treatment a therapeutically effective amount of the compound of any one of aspects 1 to 27, the granules of any one of aspects 28 to 39, the pharmaceutical composition of any one of aspects 40 to 51, or the oral dosage form of any one of aspects 52 to 61.

Aspect 70. The method of aspect 69, wherein the disease is cataplexy associated with narcolepsy.

Aspect 71. The method of aspect 69, wherein the disease is excessive daytime sleepiness associated with narcolepsy.

Aspect 72. The method of aspect 69, wherein the disease is excessive daytime sleepiness in a patient with Parkinson's disease.

Aspect 73. The method of aspect 69, wherein the disease is chronic fatigue in a patient with Parkinson's disease.

Aspect 74. A method of treating a symptom associated with narcolepsy, excessive daytime sleepiness, cataplexy, excessive daytime sleepiness associated with narcolepsy, excessive daytime sleepiness associated with Parkinson's disease, excessive daytime sleepiness associated with multiple sclerosis, cataplexy associated with narcolepsy, fatigue, fatigue associated with Parkinson's diseases, fatigue associated with multiple sclerosis, idiopathic hypersomnia or fibromyalgia comprising orally administering to a patient in need of such treatment a therapeutically effective amount of the compound of any one of aspects 1 to 27, the granules of any one of aspects 28 to 39, the pharmaceutical composition of any one of aspects 40 to 51, or the oral dosage form of any one of 52 to 61.

Aspect 75. A method of treating an REM sleep behavior disorder, spasmodic dystonia, schizophrenia, insomnia, insomnia associated with schizophrenia, idiopathic hypersomnia, chronic fatigue syndrome, cluster headache, Alzheimer's disease, essential tremor, post-traumatic stress syndrome, insomnia associated with post-traumatic stress syndrome, or anxiety comprising orally administering to a patient in need of such treatment a therapeutically effective amount of the compound of any one of aspects 1 to 27, the granules of any one of aspects 28 to 39, the pharmaceutical composition of any one of aspects 40 to 51, or the oral dosage form of any one of 52 to 61.

Aspect 76. A method of treating a symptom associated with REM sleep behavior disorder, spasmodic dystonia, schizophrenia, insomnia, insomnia associated with schizophrenia, idiopathic hypersomnia, chronic fatigue syndrome, cluster headache, Alzheimer's disease, essential tremor, post-traumatic stress syndrome, insomnia associated with post-traumatic stress syndrome, or anxiety comprising orally administering to a patient in need of such treatment a therapeutically effective amount of the compound of any one of aspects 1 to 27, the granules of any one of aspects 28 to 39, the pharmaceutical composition of any one of aspects 40 to 51, or the oral dosage form of any one of 52 to 61.

Aspect 77. The method of any one of aspects 66 to 76, wherein administering comprises orally administering.

Aspect 78. The method of any one of aspects 66 to 76, wherein administering comprises administering once daily (QD).

Aspect 79. The method of any one of aspects 66 to 76, wherein administering comprises administering twice daily (BID).

### EXAMPLES

The following examples describe in detail methods of preparing crystalline 4-((L-valyl)oxy)butanoic acid salts, properties of the crystalline 4-((L-valyl)oxy)butanoic acid salts, and methods of using the crystalline 4-((L-valyl)oxy)butanoic acid salts provided by the present disclosure. It will be apparent to those skilled in the art that many modifications, both to materials and methods, may be practiced without departing from the scope of the invention.

### Example 1

### Synthesis and Preparation of Crystalline (S)-4-(2-Amino-3-methylbutanoyloxy)butanoic Acid (4-((L-Valyl)oxy)butanoic Acid) Hydrochloride (1)

### Step 1: Preparation of (S)-4-hydroxybutyl 2-(tert-butoxycarbonylamino)-3-methylbutanoate

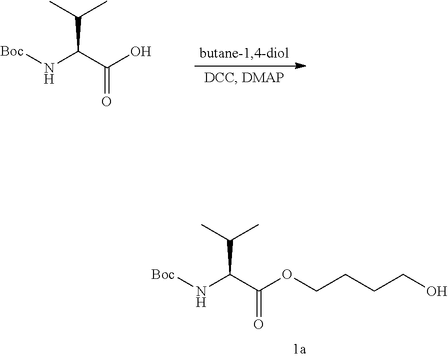 [SMILES: CC(C)[C@H](NC(=O)OC(C)(C)C)C(=O)O ; CC(C)[C@H](NC(=O)OC(C)(C)C)C(=O)OCCCCO]

(S)-2-(tert-Butoxycarbonylamino)-3-methylbutanoic acid (1 g, 4.61 mmol), N,N′-dicyclohexylcarbodiimide (DCC) (1,044 mg, 5.07 mmol) and 4-dimethylaminopyridine (DMAP) (10 mg) were added to a stirred solution of butane-1,4-diol (829 mg, 9.21 mmol) in dichloromethane (DCM) (20 mL). The reaction was stirred at 25° C. for 16 h. The reaction mixture was then diluted with saturated aqueous NH₄Cl (10 ml) and stirred for 5 min. The aqueous phase was separated and extracted with DCM (10 mL). The combined organic phase was washed with saturated brine (15 mL), dried over anhydrous Na₂SO₄, and evaporated. The residue was purified using a silica gel flash column with hexane/ethyl acetate (Hex/EA)=5:1 to yield compound (1a) (700 mg, 53% yield) as a colorless oil. ¹H NMR was performed at 400 MHz with CDCl₃ as solvent: δ=5.07 (d, J=8.8 Hz, 1H), 4.16-4.11 (m, 3H), 3.62 (t, J=6.2 Hz, 2H), 2.32 (br. s., 1H), 2.12-2.04 (m, 1H), 1.75-1.68 (m, 2H), 1.62-1.56 (m, 2H), 1.40 (s, 9H), 0.92 (d, J=7.2 Hz, 3H), 0.85 (d, J=7.2 Hz, 3H).

### Step 2: Preparation of (S)-4-(2-(tert-butoxycarbonylamino)-3-methylbutanoate

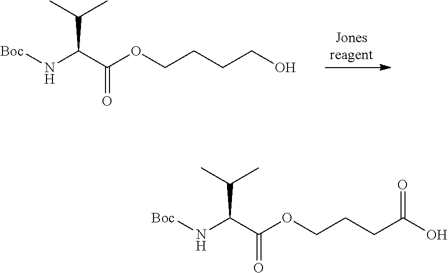 [SMILES: CC(C)[C@H](NC(=O)OC(C)(C)C)C(=O)OCCCCO ; CC(C)[C@H](NC(=O)OC(C)(C)C)C(=O)OCCCC(=O)O]

Jones reagent was added in portions to a stirred mixture of (S)-4-hydroxybutyl 2-(tert-butoxycarbonylamino)-3-methylbutanoate (1a) (500 mg, 1.73 mmol) and Celite® (diatomaceous earth, 2 g) in acetone (10 ml) at 0° C. The reaction proceeded at 0° C. for over 1 h and the reaction progress was monitored using thin layer chromatography (TLC). After completion, the reaction was quenched with drops of isopropanol, diluted with ethyl acetate (EA) (10 ml) and then filtered. The filter cake was washed with EA (5 ml) and the combined filtrate was washed with saturated brine (2 mL×2), dried over anhydrous Na₂SO₄, and concentrated. The residue was purified using a silica gel flash column with Hex/EA=10:1-5:1 to yield compound (1b) (170 mg, 32% yield) as a white solid. ¹H NMR was performed at 400 MHz with CDCl₃ as solvent: δ=5.03 (d, J=9.2 Hz, 1H), 4.30-4.24 (m, 1H), 4.22-4.13 (m, 2H), 2.46 (t, J=7.4 Hz, 2H), 2.16-2.08 (m, 1H), 2.06-1.96 (m, 2H), 1.45 (s, 9H), 0.96 (d, J=6.8 Hz, 3H), 0.89 (d, J=6.4 Hz, 3H).

### Step 3: Preparation of crystalline (S)-4-(2-amino-3-methylbutanoyloxy)butanoic acid hydrochloride

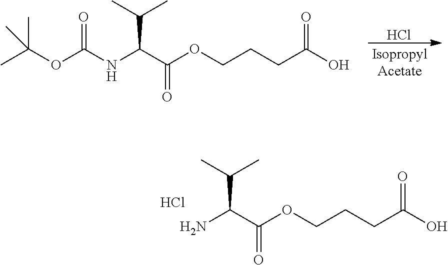 [SMILES: CC(C)[C@H](NC(=O)OC(C)(C)C)C(=O)OCCCC(=O)O ; Cl ; Cl ; CC(C)[C@H](N)C(=O)OCCCC(=O)O]

(S)-4-Hydroxybutyl 2-(tert-butoxycarbonylamino)-3-methylbutanoate (1a) (2.00 g, 6.60 mmol, solid) and HCl (gas) in isopropyl acetate (20 mL, 4 M) were added into and stirred in a one-neck round flask, and then cooled with ice-water bath. The reaction turned into a clear solution. The mixture was gradually warmed to 25° C. and stirred for 16 hours. White precipitates formed. The reaction mixture was filtered to collect the white precipitates which were washed with isopropyl acetate (10 mL) to give crude solids. The crude solids and isopropyl acetate (5 mL) were added in a flask and stirred at 25° C. for 1 hour. The mixture was filtered and the filter cake washed with isopropyl acetate (5 mL) and dried over vacuum to give the crystalline (S)-4-(2-amino-3-methylbutanoyloxy)butanoic acid (4-((L-valyl)oxy)butanoic acid) hydrochloride salt (1.10 g, yield: 62%, purity: 98.77%) as a white crystal. ¹H NMR (400 MHz, Methanol-d4) δ=4.36-4.26 (m, 2H), 3.94 (d, J=4.7 Hz, 1H), 2.43 (t, J=7.2 Hz, 2H), 2.30 (dt, J=4.7, 7.0 Hz, 1H), 2.05-1.94 (m, 2H), 1.08 (d, J=7.0 Hz, 6H). Melting point: 99.2° C.±0.5° C. as determined by differential scanning calorimetry.

Crystalline 4-((L-valyl)oxy)butanoic acid hydrochloride had a compositional purity of about 98.8% determined by HPLC using a Thermo Fischer Scientific U3000 instrument equipped with a Waters Atlantis® T3 (5 μm, 4.6×150 mm) column with a mobile phase consisting of (A) water containing 0.15% phosphoric acid and (B) acetonitrile, and a chiral purity of about 100% determined by SFC (supercritical fluid chromatography).

### Example 2

### Preparation of Crystalline (S)-4-(2-Amino-3-methylbutanoyloxy)butanoic Acid (4-((L-Valyl)oxy)butanoic Acid) Oxalate Salt (2)

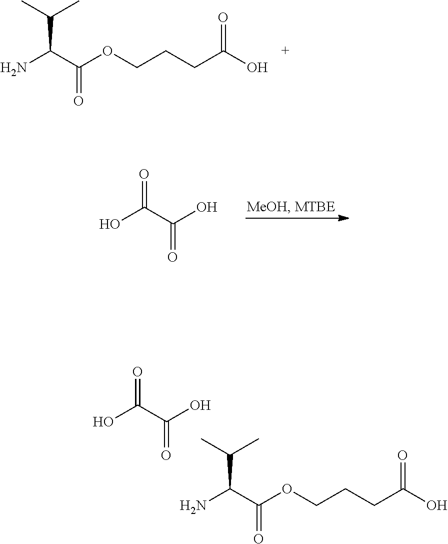

Oxalic acid (221 mg, 2.46 mmol, 1 equiv) was added to 4-((L-valyl)oxy)butanoic acid (500 mg, 2.46 mmol, 1 equiv) in MeOH (3.5 mL) and the reaction mixture was stirred at 25° C. for 3 hours. Methyl tert-butyl ether (MTBE, 25 mL) was added into the mixture and white precipitates were gradually formed. The reaction mixture was stirred at 25° C. for an additional 16 hours. After that, the reaction mixture was filtered to collect the white precipitates which were washed with MTBE (15 mL) to give wet crude solids. The wet solids and MTBE (10 mL) were added into a flask, the mixture was stirred at 25° C. for 1 hour. The mixture was filtered and the filter cake was washed with MTBE (5 mL) and dried over vacuum to give the oxalate salt (630 mg, yield: 87%, purity: 99.42%) as a white crystal. The ratio of oxalic acid to 4-((L-valyl)oxy)butanoic acid is 1.2 to 1 was determined using HPLC. ¹H NMR (400 MHz, methanol-d4) δ=4.36-4.24 (m, 2H), 3.93 (d, J=4.7 Hz, 1H), 2.43 (t, J=7.2 Hz, 2H), 2.30 (qd, J=6.9, 11.7 Hz, 1H), 2.04-1.94 (m, 2H), 1.07 (d, J=6.7 Hz, 3H), 1.08 (d, J=6.7 Hz, 3H). ¹³C NMR (101 MHz, methanol-d4) δ=176.7, 170.2, 166.7, 66.7, 59.6, 31.4, 31.1, 25.2, 18.6, 18.4. Melting point: 143.3° C.±0.5° C. as determined by differential scanning calorimetry.

Crystalline 4-((L-valyl)oxy)butanoic acid oxalate had a compositional purity of about 99.4% and a chiral purity of about 100% as determined by HPLC using a Thermo Fischer Scientific U3000 instrument equipped with a Waters Atlantis® T3 (5 μm, 4.6×150 mm) column with a mobile phase consisting of (A) water containing 0.15% phosphoric acid and (B) acetonitrile.

### Example 3

### Synthesis of 4-((L-Valyl)oxy)butanoic Acid Mesylate, Trifluoroacetate, and p-Toluenesulfonate Salts

To a stirred solution of 4-((L-valyl)oxy)butanoic acid (500 mg, 2.46 mmol, 1 equiv) in MeOH (3.5 mL) was added with a suitable acid (1 equiv) such as methanesulfonic acid, trichloroacetic acid, or p-toluenesulfonic acid monohydrate. The reaction was stirred at 25° C. for 3 hours. The reaction was then concentrated to dryness to give the desired 4-((L-valyl)oxy)butanoic acid salts.

4-((L-Valyl)oxy)butanoic acid mesylate salt (763 mg, colorless oil) ¹H NMR (400 MHz, methanol-d4) δ=4.37-4.24 (m, 2H), 3.94 (d, J=4.3 Hz, 1H), 2.70 (s, 3H), 2.43 (t, J=7.2 Hz, 2H), 2.30 (dt, J=4.7, 7.0 Hz, 1H), 2.05-1.95 (m, 2H), 1.08 (dd, J=1.8, 6.8 Hz, 6H).

4-((L-Valyl)oxy)butanoic acid trifluoroacetate salt (780 mg, colorless oil) ¹H NMR (400 MHz, methanol-d4) δ=4.36-4.24 (m, 2H), 3.92 (d, J=4.3 Hz, 1H), 2.42 (t, J=7.2 Hz, 2H), 2.30 (qd, J=6.8, 11.7 Hz, 1H), 2.00 (quin, J=6.9 Hz, 2H), 1.07 (dd, J=2.0, 7.0 Hz, 6H).

4-((L-Valyl)oxy)butanoic acid p-toluenesulfonate salt (923 mg, white waxy solid) ¹H NMR (400 MHz, methanol-d4) δ=7.75-7.66 (m, J=8.2 Hz, 2H), 7.29-7.20 (m, J=7.8 Hz, 2H), 4.35-4.23 (m, 2H), 3.92 (d, J=4.3 Hz, 1H), 2.42 (t, J=7.4 Hz, 2H), 2.37 (s, 3H), 2.29 (qd, J=6.9, 11.7 Hz, 1H), 1.99 (quin, J=6.9 Hz, 2H), 1.06 (dd, J=1.6, 7.0 Hz, 6H).

### Example 4

### X-Ray Powder Diffraction (XPRD) of Crystalline 4-((L-Valyl)oxy)butanoic Acid Salts

The X-ray powder diffraction (XRPD) pattern of crystalline 4-((L-valyl)oxy)butanoic acid hydrochloride and crystalline 4-((L-valyl)oxy)butanoic acid oxalate were obtained using a Bruker D8 Advance X-ray powder diffractometer. The 20 position was calibrated against a Malvern Panalytical Si standard disc. The X-ray wavelength was Kα2/Kα1 (1.540598 Å/1.544425 Å) at a 0.5 intensity ratio. The X-ray tube was set at an output voltage of 40 kV and a current of 40 mA. A ⅛° fixed divergence slit was used, and the diffraction patterns were obtained from 3° to 40° (°2θ) in a continuous scan mode using a step size of 0.02° (2θ) and a scan speed of 0.145 deg/min. The samples were transferred from a sample container onto a zero background XRPD-holder and gently ground to provide a smooth surface.

A representative XRPD diffraction pattern of crystalline 4-((L-valyl)oxy)butanoic acid hydrochloride and crystalline 4-((L-valyl)oxy)butanoic acid oxalate is shown in FIGS. 1 and 3, respectively, and the characteristic diffraction peaks are listed in Tables 1 and 2, respectively.

TABLE 1
Characteristic peaks of the XRPD pattern for crystalline
4-((L-valyl)oxy)butanoic acid hydrochloride.

| Peak Index | Angle (°2θ) | Relative Intensity (%) |
|---|---|---|
| 1 | 7.24 | 0.6 |
| 2 | 8.05 | 60.5 |
| 3 | 10.07 | 5.5 |
| 4 | 10.72 | 25.0 |
| 5 | 16.12 | 36.5 |
| 6 | 16.53 | 17.1 |
| 7 | 17.62 | 17.4 |
| 8 | 18.00 | 4.9 |
| 9 | 19.50 | 0.6 |
| 10 | 20.22 | 11.3 |
| 11 | 20.72 | 3.7 |
| 12 | 21.66 | 100.0 |
| 13 | 23.14 | 5.5 |
| 14 | 24.09 | 73.0 |
| 15 | 24.94 | 3.9 |
| 16 | 26.03 | 16.0 |
| 17 | 27.38 | 25.8 |
| 18 | 28.35 | 9.8 |
| 19 | 29.10 | 6.8 |
| 20 | 29.69 | 10.1 |
| 21 | 30.09 | 19.1 |
| 22 | 30.85 | 6.8 |
| 23 | 31.09 | 3.7 |
| 24 | 32.51 | 4.8 |
| 25 | 32.72 | 5.7 |
| 26 | 33.41 | 6.2 |
| 27 | 33.92 | 6.6 |
| 28 | 35.12 | 2.4 |
| 29 | 35.69 | 9.5 |
| 30 | 36.50 | 3.0 |
| 31 | 37.16 | 3.0 |
| 32 | 38.11 | 2.2 |
| 33 | 38.93 | 2.0 |
| 34 | 39.58 | 1.8 |

TABLE 2
Characteristic peaks of the XRPD pattern for crystalline
4-((L-valyl)oxy)butanoic acid oxalate.

| Peak Index | Angle (°2θ) |
|---|---|
| 1 | 6.48 |
| 2 | 9.44 |
| 3 | 11.04 |
| 4 | 17.03 |
| 5 | 18.20 |
| 6 | 18.73 |
| 7 | 19.08 |
| 8 | 21.73 |
| 9 | 22.46 |
| 10 | 27.24 |
| 11 | 28.86 |
| 12 | 29.45 |

### Example 5

### Differential Scanning Calorimetry Profile of Crystalline 4-((L-Valyl)oxy)butanoic Acid Salts

A differential scanning calorimetry profile of crystalline 4-((L-valyl)oxy)butanoic acid hydrochloric acid is shown in FIG. 2. The DSC profile from 40° C. to 130° C. exhibits and exothermic onset temperature of 97.04° C., and a peak temperature of 99.19° C.

### Example 6

### Aqueous Stability of Crystalline 4-((L-Valyl)oxy)butanoic Acid Salts

A shake flask method followed by HPLC analysis was used to determine the aqueous stability of the crystalline 4-((L-valyl)oxy)butanoic acid salts.

Test solutions were prepared by adding 100 μL (10 μL) stock solution (100 mg/ml crystalline 4-((L-valyl)oxy)butanoic acid salt) to preheated DMSO or to buffer solution (pH 1.2, 2.0, 4.0, 6.0, 8.0) to give a final concentration of 10 mg/ml or 1 mg/mL.

The test solutions were incubated at 37° C. for up to 240 hours and the amount of crystalline 4-((L-valyl)oxy)butanoic acid salt was determined by LC-MS using a Shimadzu LC-MS2020 equipped with a Chromolith® Flash RP-18e (1 μm 2×25 mm) column and using a mobile phase consisting of a combination of water containing 0.05% TFA and acetonitrile.

The pH 1.2 HCl buffer solution was prepared by diluting 765 μL of concentrated HCl with water to a volume of 100 mL in a volumetric flask.

The pH 2.0 HCl buffer was prepared by diluting 117 μL of concentrated HCl with water to a volume of 100 mL in a volumetric flask.

The pH 4.0 acetate buffer was prepared by diluting 12 g (11.4 mL) of acetic acid with water to a volume of 100 mL to generate a 2 mol/L acetic acid solution. 122 mg of sodium acetate was mixed with the 2.05 ml the 2 mol/L acetic acid solution, and the mixture diluted with water to a volume of 100 mL in a volumetric flask.

The pH 6.0 phosphate buffer was prepared by dissolving 2.722 g KH₂PO₄ in water and diluting with water to a volume of 100 mL in a volumetric flask to provide a 0.2 mol/L solution (solution A). 0.8 g NaOH was dissolved in water and diluted to a volume of 100 mL in a volumetric flask to provide a 0.2 mol/L solution (solution B).

The pH 8.0 phosphate buffer solution was prepared by mixing 25 mL the solution A and 23.05 mL of solution B and diluting the mixture with water to a volume of 100 mL in a volumetric flask.

The crystalline 4-((L-valyl)oxy)butanoic acid salt stock solution was prepared by dissolving 1 g of crystalline 4-((L-valyl)oxy)butanoic acid salt in DMSO, and then diluting to a volume of 10 mL in a volumetric flask to provide a 100 mg/mL crystalline 4-((L-valyl)oxy)butanoic acid salt stock solution.

The stability results for 10 mg/mL and 1 mg/mL crystalline 4-((L-valyl)oxy)butanoic acid hydrochloride solutions at various pH and in DMSO are presented in the Table 3.

TABLE 3
Stability of crystalline 4-((L-valyl)oxy)butanoic acid hydrochloride.

| | | Concentration | Concentration |
|---|---|---|---|
| | Time | 10 mg/mL | 1 mg/mL |
| pH | (hours) | (assay %) | (assay %) |
| 1.2 | 240 | 99.6, 100.3 | 100, 100 |
| 2.0 | 240 | 99.9, 99.9 | 92, 96.4 |
| 4.0 | 240 | 95.5, 95.7 | 95, 93 |
| 6.0 | 240 | 97.9, 100 | 98.4, 98 |
| 8.0 | 240 | 100, 100 | 99.2, 103.2 |
| DMSO | 96 | 98 | 105 |

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

What is claimed is:

1. A compound selected from crystalline 4-((L-valyl)oxy)butanoic acid hydrochloride and crystalline 4-((L-valyl)oxy)butanoic acid oxalate.

2. The compound of claim 1, wherein the compound is crystalline 4-((L-valyl)oxy)butanoic acid hydrochloride having the structure of Formula (1):

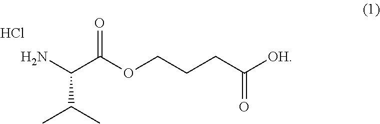

3. The compound of claim 2, wherein the compound is characterized by an XRPD pattern comprising characteristic diffraction peaks at least at 8.05°±0.20°, 21.66°±0.20°, and 24.09°±0.20° expressed as 2θ/θ angles determined using Cu-Kα radiation.

4. The compound of claim 2, wherein the compound has a DSC profile characterized by an exotherm with an onset temperature at 97.04° C.±2° C., and a peak at 99.19° C.±2° C.

5. The compound of claim 1, wherein the compound is crystalline 4-((L-valyl)oxy)butanoic acid oxalate having the structure of Formula (2):

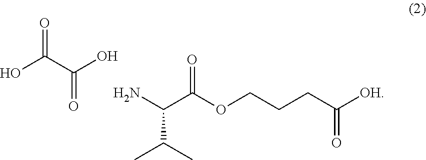

6. The compound of claim 5, wherein the compound is characterized by an XRPD pattern comprising characteristic diffraction peaks at least at 6.48°±0.20°, 9.44°±0.20°, 18.20°±0.20°, and 27.24°±0.20°, expressed as 2θ/θ angles determined using Cu-Kα radiation.

7. Granules comprising the compound of claim 1.

8. The granules of claim 7, wherein the granules have an average particle size from 100 μm to 600 μm.

9. The granules of claim 7, wherein the granules comprise a plurality of immediate release granules.

10. The granules of claim 7, wherein the granules comprise a plurality of modified release granules.

11. A pharmaceutical composition comprising the compound of claim 1.

12. The pharmaceutical composition of claim 11, wherein the pharmaceutical composition comprises a therapeutically effective amount of the compound for treating a disease in a patient, wherein the disease is selected from narcolepsy, cataplexy, cataplexy with narcolepsy, excessive daytime sleepiness, a sleep disorder associated with Parkinson's disease, Parkinson's disease, a neurodegenerative disease, idiopathic hypersomnia, disturbed nocturnal sleep, sleep disturbance syndrome, fatigue, improving nocturnal sleep, hypnagogic hallucinations, sleep paralysis, fragmented sleep, alcohol withdrawal and dependence, obstructive sleep apnea syndrome, insomnia, insomnia associated with schizophrenia, sleep ignition and maintenance disorders, and chronic fatigue syndrome.

13. The pharmaceutical composition of claim 11, wherein the pharmaceutical composition comprises an immediate release component.

14. The pharmaceutical composition of claim 13, wherein the immediate release component comprises immediate release granules.

15. The pharmaceutical composition of claim 13, wherein the immediate release component comprises a solution comprising the compound.

16. The pharmaceutical composition of claim 11, wherein the pharmaceutical composition comprises a modified release component.

17. The pharmaceutical composition of claim 16, wherein the modified release component comprises modified release granules.

18. The pharmaceutical composition of claim 16, wherein the modified release component comprises modified release granules suspended in a viscous aqueous solution.

19. A kit comprising the compound of claim 1.

20. A method of treating a disease in a patient comprising administering to a patient in need of such treatment a therapeutically effective amount of the compound of claim 1.
